SEQUENCE LISTING (44 sequences)

SEQ ID NO: 1 - DNA, 64 bp, Artificial Sequence
  Description of Artificial Sequence Synthetic primer
ggggacaagtttgtacaaaaaagcaggctccatgaacactccaaaagaagaattccaggactgg

SEQ ID NO: 2 - DNA, 69 bp, Artificial Sequence
  Description of Artificial Sequence Synthetic primer
ggggaccactttgtacaagaaagctgggttcagtggtggtggtggtggtggactttctcagtacggccc

SEQ ID NO: 3 - protein, 475 aa, Artificial Sequence
  Description of Artificial Sequence Synthetic polypeptide
MNTPKEEFQDWPIVRIAAHLPDLIVYGHFSPERPFMDYFDGVLMFVDISGFTAMTEKFSSAMYMDRGAEQLVEILNYHISAIVEKVLIFGGDILKFAGDALLALWRVERKQLKNIITVVIKCSLEIHGLFETQEWEEGLDIRVKIGLAAGHISMLVFGDETHSHFLVIGQAVDDVRLAQNMAQMNDVILSPNCWQLCDRSMIEIESVPDQRAVKVNFLKPPPNFNFDEFFTKCTTFMHYYPSGEHKNLLRLACTLKPDPELEMSLQKYVMESILKQIDNKQLQGYLSELRPVTIVFVNLMFEDQDKAEEIGPAIQDAYMHITSVLKIFQGQINKVFMFDKGCSFLCVFGFPGEKVPDELTHALECAMDIFDFCSQVHKIQTVSIGVASGIVFCGIVGHTVRHEYTVIGQKVNLAARMMMYYPGIVTCDSVTYNGSNLPAYFFKELPKKVMKGVADSGPLYQYWGRTEKVHHHHHH

SEQ ID NO: 4 - protein, 6 aa, Artificial Sequence
  Description of Artificial Sequence Synthetic 6xHis tag
HHHHHH

SEQ ID NO: 5 - protein, 246 aa, Artificial Sequence
  Description of Artificial Sequence Synthetic polypeptide
MNTPKEEFQDWPIVRIAAHLPDLIVYGHFSPERPFMDYFDGVLMFVDISGFTAMTEKFSSAMYMDRGAEQLVEILNYHISAIVEKVLIFGGDILKFAGDALLALWRVERKQLKNIITVVIKCSLEIHGLFETQEWEEGLDIRVKIGLAAGHISMLVFGDETHSHFLVIGQAVDDVRLAQNMAQMNDVILSPNCWQLCDRSMIEIESVPDQRAVKVNFLKPPPNFNFDEFFTKCTTFMHYYPSGEHK

SEQ ID NO: 6 - protein, 222 aa, Artificial Sequence
  Description of Artificial Sequence Synthetic polypeptide
NLLRLACTLKPDPELEMSLQKYVMESILKQIDNKQLQGYLSELRPVTIVFVNLMFEDQDKAEEIGPAIQDAYMHITSVLKIFQGQINKVFMFDKGCSFLCVFGFPGEKVPDELTHALECAMDIFDFCSQVHKIQTVSIGVASGIVFCGIVGHTVRHEYTVIGQKVNLAARMMMYYPGIVTCDSVTYNGSNLPAYFFKELPKKVMKGVADSGPLYQYWGRTEK

SEQ ID NO: 7 - protein, 26 aa, Artificial Sequence
  Description of Artificial Sequence Synthetic peptide
MNTPKEEFQDWPIVRIAAHLPDLIVY

SEQ ID NO: 8 - protein, 66 aa, Artificial Sequence
  Description of Artificial Sequence Synthetic peptide
KPPPNFNFDEFFTKCTTFMHYYPSGEHKNLLRLACTLKPDPELEMSLQKYVMESILKQIDNKQLQG

SEQ ID NO: 9 - protein, 7 aa, Artificial Sequence
  Description of Artificial Sequence Synthetic peptide
IVRIAAH

SEQ ID NO: 10 - protein, 11 aa, Artificial Sequence
  Description of Artificial Sequence Synthetic peptide
FDEFFTKCTTF

SEQ ID NO: 11 - protein, 11 aa, Artificial Sequence
  Description of Artificial Sequence Synthetic peptide
DPELEMSLQKY

SEQ ID NO: 12 - protein, 7 aa, Artificial Sequence
  Description of Artificial Sequence Synthetic peptide
ESILKQI

SEQ ID NO: 13 - protein, 4 aa, Artificial Sequence
  Description of Artificial Sequence Synthetic 4xHis tag
HHHH

SEQ ID NO: 14 - protein, 5 aa, Artificial Sequence
  Description of Artificial Sequence Synthetic 5xHis tag
HHHHH

SEQ ID NO: 15 - protein, 7 aa, Artificial Sequence
  Description of Artificial Sequence Synthetic 7xHis tag
HHHHHHH

SEQ ID NO: 16 - protein, 8 aa, Artificial Sequence
  Description of Artificial Sequence Synthetic 8xHis tag
HHHHHHHH

SEQ ID NO: 17 - protein, 10 aa, Artificial Sequence
  Description of Artificial Sequence Synthetic His tag
HHHHHHHHHH

SEQ ID NO: 18 - protein, 6 aa, Artificial Sequence
  Description of Artificial Sequence Synthetic peptide
WEEGLD

SEQ ID NO: 19 - protein, 7 aa, Artificial Sequence
  Description of Artificial Sequence Synthetic peptide
FPGEKVP

SEQ ID NO: 20 - protein, 5 aa, Artificial Sequence
  Description of Artificial Sequence Synthetic peptide
KGVAD

SEQ ID NO: 21 - protein, 181 aa, Artificial Sequence
  Description of Artificial Sequence Synthetic polypeptide
MDYFDGVLMFVDISGFTAMTEKFSSAMYMDRGAEQLVEILNYHISAIVEKVLIFGGDILKFAGDALLALWRVERKQLKNIITVVIKCSLEIHGLFETQEWEEGLDIRVKIGLAAGHISMLVFGDETHSHFLVIGQAVDDVRLAQNMAQMNDVILSPNCWQLCDRSMIEIESVPDQRAVKVN

SEQ ID NO: 22 - protein, 179 aa, Artificial Sequence
  Description of Artificial Sequence Synthetic polypeptide
SELRPVTIVFVNLMFEDQDKAEEIGPAIQDAYMHITSVLKIFQGQINKVFMFDKGCSFLCVFGFPGEKVPDELTHALECAMDIFDFCSQVHKIQTVSIGVASGIVFCGIVGHTVRHEYTVIGQKVNLAARMMMYYPGIVTCDSVTYNGSNLPAYFFKELPKKVMKGVADSGPLYQYWGR

SEQ ID NO: 23 - protein, 12 aa, Artificial Sequence
  Description of Artificial Sequence Synthetic peptide
MNTPKEEFQDWP

SEQ ID NO: 24 - protein, 7 aa, Artificial Sequence
  Description of Artificial Sequence Synthetic peptide
LPDLIVY

SEQ ID NO: 25 - protein, 469 aa, Artificial Sequence
  Description of Artificial Sequence Synthetic polypeptide
MNTPKEEFQDWPIVRIAAHLPDLIVYGHFSPERPFMDYFDGVLMFVDISGFTAMTEKFSSAMYMDRGAEQLVEILNYHISAIVEKVLIFGGDILKFAGDALLALWRVERKQLKNIITVVIKCSLEIHGLFETQEWEEGLDIRVKIGLAAGHISMLVFGDETHSHFLVIGQAVDDVRLAQNMAQMNDVILSPNCWQLCDRSMIEIESVPDQRAVKVNFLKPPPNFNFDEFFTKCTTFMHYYPSGEHKNLLRLACTLKPDPELEMSLQKYVMESILKQIDNKQLQGYLSELRPVTIVFVNLMFEDQDKAEEIGPAIQDAYMHITSVLKIFQGQINKVFMFDKGCSFLCVFGFPGEKVPDELTHALECAMDIFDFCSQVHKIQTVSIGVASGIVFCGIVGHTVRHEYTVIGQKVNLAARMMMYYPGIVTCDSVTYNGSNLPAYFFKELPKKVMKGVADSGPLYQYWGRTEKV

SEQ ID NO: 26 - protein, 457 aa, Artificial Sequence
  Description of Artificial Sequence Synthetic polypeptide
IVRIAAHLPDLIVYGHFSPERPFMDYFDGVLMFVDISGFTAMTEKFSSAMYMDRGAEQLVEILNYHISAIVEKVLIFGGDILKFAGDALLALWRVERKQLKNIITVVIKCSLEIHGLFETQEWEEGLDIRVKIGLAAGHISMLVFGDETHSHFLVIGQAVDDVRLAQNMAQMNDVILSPNCWQLCDRSMIEIESVPDQRAVKVNFLKPPPNFNFDEFFTKCTTFMHYYPSGEHKNLLRLACTLKPDPELEMSLQKYVMESILKQIDNKQLQGYLSELRPVTIVFVNLMFEDQDKAEEIGPAIQDAYMHITSVLKIFQGQINKVFMFDKGCSFLCVFGFPGEKVPDELTHALECAMDIFDFCSQVHKIQTVSIGVASGIVFCGIVGHTVRHEYTVIGQKVNLAARMMMYYPGIVTCDSVTYNGSNLPAYFFKELPKKVMKGVADSGPLYQYWGRTEKV

SEQ ID NO: 27 - protein, 434 aa, Artificial Sequence
  Description of Artificial Sequence Synthetic polypeptide
MDYFDGVLMFVDISGFTAMTEKFSSAMYMDRGAEQLVEILNYHISAIVEKVLIFGGDILKFAGDALLALWRVERKQLKNIITVVIKCSLEIHGLFETQEWEEGLDIRVKIGLAAGHISMLVFGDETHSHFLVIGQAVDDVRLAQNMAQMNDVILSPNCWQLCDRSMIEIESVPDQRAVKVNFLKPPPNFNFDEFFTKCTTFMHYYPSGEHKNLLRLACTLKPDPELEMSLQKYVMESILKQIDNKQLQGYLSELRPVTIVFVNLMFEDQDKAEEIGPAIQDAYMHITSVLKIFQGQINKVFMFDKGCSFLCVFGFPGEKVPDELTHALECAMDIFDFCSQVHKIQTVSIGVASGIVFCGIVGHTVRHEYTVIGQKVNLAARMMMYYPGIVTCDSVTYNGSNLPAYFFKELPKKVMKGVADSGPLYQYWGRTEKV

SEQ ID NO: 28 - protein, 443 aa, Artificial Sequence
  Description of Artificial Sequence Synthetic polypeptide
GHFSPERPFMDYFDGVLMFVDISGFTAMTEKFSSAMYMDRGAEQLVEILNYHISAIVEKVLIFGGDILKFAGDALLALWRVERKQLKNIITVVIKCSLEIHGLFETQEWEEGLDIRVKIGLAAGHISMLVFGDETHSHFLVIGQAVDDVRLAQNMAQMNDVILSPNCWQLCDRSMIEIESVPDQRAVKVNFLKPPPNFNFDEFFTKCTTFMHYYPSGEHKNLLRLACTLKPDPELEMSLQKYVMESILKQIDNKQLQGYLSELRPVTIVFVNLMFEDQDKAEEIGPAIQDAYMHITSVLKIFQGQINKVFMFDKGCSFLCVFGFPGEKVPDELTHALECAMDIFDFCSQVHKIQTVSIGVASGIVFCGIVGHTVRHEYTVIGQKVNLAARMMMYYPGIVTCDSVTYNGSNLPAYFFKELPKKVMKGVADSGPLYQYWGRTEKV

SEQ ID NO: 29 - protein, 245 aa, Artificial Sequence
  Description of Artificial Sequence Synthetic polypeptide
MNTPKEEFQDWPIVRIAAHLPDLIVYGHFSPERPFMDYFDGVLMFVDISGFTAMTEKFSSAMYMDRGAEQLVEILNYHISAIVEKVLIFGGDILKFAGDALLALWRVERKQLKNIITVVIKCSLEIHGLFETQEWEEGLDIRVKIGLAAGHISMLVFGDETHSHFLVIGQAVDDVRLAQNMAQMNDVILSPNCWQLCDRSMIEIESVPDQRAVKVNFLKPPPNFNFDEFFTKCTTFMHYYPSGEH

SEQ ID NO: 30 - protein, 215 aa, Artificial Sequence
  Description of Artificial Sequence Synthetic polypeptide
LKPDPELEMSLQKYVMESILKQIDNKQLQGYLSELRPVTIVFVNLMFEDQDKAEEIGPAIQDAYMHITSVLKIFQGQINKVFMFDKGCSFLCVFGFPGEKVPDELTHALECAMDIFDFCSQVHKIQTVSIGVASGIVFCGIVGHTVRHEYTVIGQKVNLAARMMMYYPGIVTCDSVTYNGSNLPAYFFKELPKKVMKGVADSGPLYQYWGRTEKV

SEQ ID NO: 31 - protein, 11 aa, Artificial Sequence
  Description of Artificial Sequence Synthetic peptide
ISGFTAMTEKF

SEQ ID NO: 32 - protein, 6 aa, Artificial Sequence
  Description of Artificial Sequence Synthetic peptide
FAGDAL

SEQ ID NO: 33 - protein, 4 aa, Artificial Sequence
  Description of Artificial Sequence Synthetic peptide
AGDA

SEQ ID NO: 34 - protein, 9 aa, Artificial Sequence
  Description of Artificial Sequence Synthetic peptide
VFMFDKGCS

SEQ ID NO: 35 - protein, 4 aa, Artificial Sequence
  Description of Artificial Sequence Synthetic peptide
FLVI

SEQ ID NO: 36 - protein, 4 aa, Artificial Sequence
  Description of Artificial Sequence Synthetic peptide
VFMF

SEQ ID NO: 37 - protein, 4 aa, Artificial Sequence
  Description of Artificial Sequence Synthetic peptide
FDKG

SEQ ID NO: 38 - protein, 134 aa, Artificial Sequence
  Description of Artificial Sequence Synthetic polypeptide
MNTPKEEFQDWPIVRIAAHLPDLIVYGHFSPERPFMDYFDGVLMFVDISGFTAMTEKFSSAMYMDRGAEQLVEILNYHISAIVEKVLIFGGDILKFAGDALLALWRVERKQLKNIITVVIKCSLEIHGLFETQE

SEQ ID NO: 39 - protein, 209 aa, Artificial Sequence
  Description of Artificial Sequence Synthetic polypeptide
IRVKIGLAAGHISMLVFGDETHSHFLVIGQAVDDVRLAQNMAQMNDVILSPNCWQLCDRSMIEIESVPDQRAVKVNFLKPPPNFNFDEFFTKCTTFMHYYPSGEHKNLLRLACTLKPDPELEMSLQKYVMESILKQIDNKQLQGYLSELRPVTIVFVNLMFEDQDKAEEIGPAIQDAYMHITSVLKIFQGQINKVFMFDKGCSFLCVFG

SEQ ID NO: 40 - protein, 112 aa, Artificial Sequence
  Description of Artificial Sequence Synthetic polypeptide
DELTHALECAMDIFDFCSQVHKIQTVSIGVASGIVFCGIVGHTVRHEYTVIGQKVNLAARMMMYYPGIVTCDSVTYNGSNLPAYFFKELPKKVMKGVADSGPLYQYWGRTEK

SEQ ID NO: 41 - protein, 210 aa, Artificial Sequence
  Description of Artificial Sequence Synthetic polypeptide
IRVKIGLAAGHISMLVFGDETHSHFLVIGQAVDDVRLAQNMAQMNDVILSPNCWQLCDRSMIEIESVPDQRAVKVNFLKPPPNFNFDEFFTKCTTFMHYYPSGEHKNLLRLACTLKPDPELEMSLQKYVMESILKQIDNKQLQGYLSELRPVTIVFVNLMFEDQDKAEEIGPAIQDAYMHITSVLKIFQGQINKVFMFDKGCSFLCVFGF

SEQ ID NO: 42 - protein, 470 aa, Artificial Sequence
  Description of Artificial Sequence Synthetic polypeptide
MNTPKEEFQDWPIVRIAAHLPDLIVYGHFSPERPFMDYFDGVLMFVDISGFTAMTEKFSSAMYMDRGAEQLVEILNYHISAIVEKVLIFGGDILKFAGDALLALWRVERKQLKNIITVVIKCSLEIHGLFETQEWEEGLDIRVKIGLAAGHISMLVFGDETHSHFLVIGQAVDDVRLAQNMAQMNDVILSPNCWQLCDRSMIEIESVPDQRAVKVNFLKPPPNFNFDEFFTKCTTFMHYYPSGEHKNLLRLACMETLKPDPELEMSLQKYVMESILKQIDNKQLQGYLSELRPVTIVFVNLMFEDQDKAEEIGPAIQDAYMHITSVLKIFQGQINKVFMFDKGCSFLCVFGFPGEKVPDELTHALECAMDIFDFCSQVHKIQTVSIGVASGIVFCGIVGHTVRHEYTVIGQKVNLAARMMMYYPGIVTCDSVTYNGSNLPAYFFKELPKKVMKGVADSGPLYQYWGRTEK

SEQ ID NO: 43 - protein, 6 aa, Artificial Sequence
  Description of Artificial Sequence Synthetic peptide
DISGFT

SEQ ID NO: 44 - protein, 4 aa, Artificial Sequence
  Description of Artificial Sequence Synthetic peptide
KNLL

(19) United States
(12) Patent Application Publication
(10) Pub. No.: US 2009/0275047 A1
(43) Pub. Date: Nov. 5, 2009

(54) CRYSTAL STRUCTURE OF HUMAN SOLUBLE ADENYLATE CYCLASE

(76) Inventors: Susanne Maria Saalau-Bethell, Cambridgeshire (GB); Anne Cleasby, Cambridgeshire (GB); Joseph Coyle, Cambridgeshire (GB); Mladen Vinkovic, Cambridgeshire (GB); Tracey Ann Sambrook, Cambridgeshire (GB)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/996,187

(22) PCT Filed: Jul. 21, 2006
(86) PCT No.: PCT/GB06/02750

§ 371 Date: Jan. 18, 2008

Related U.S. Application Data
(60) Provisional application No. 60/701,122, filed on Jul. 21, 2005.

(51) Int. Cl. G01N 33/53; G06G 7/58; C12N 9/00

(52) U.S. Cl. 435/7.1; 703/11; 435/183

(57) ABSTRACT

The invention provides the crystal structure of the solAC catalytic domain. The structure is set out in Tables 1 to 5. The structure may be used in to model the interaction of ligands such as pharmaceutical compounds with this protein, and to determine the structure of related adenylate cyclase molecules.

### FIELD OF THE INVENTION

The present invention relates to the catalytic domain of human soluble adenylate cyclase (solAC), methods for its crystallization, crystals of solAC and their 3-dimensional structures, crystals of solAC in the presence of ligand, and uses thereof.

### BACKGROUND TO THE INVENTION

### Adenylate Cyclases

Cyclic adenosine 3′,5′-monophosphate (cAMP) is a ubiquitous second messenger which regulates a number of essential physiological processes including gene expression, cell growth, cardiac function, chromosome segregation and cellular metabolism (Robison, G. A. Butcher, R. W. and Sutherland, E. W. (1968) Annu. Rev. Biochem., 37, 149-174; Rodbell, M. (1980) Nature, 284, 17-22). It is synthesised from ATP by six different families of enzymes, one of which is the adenylate or adenylyl cyclases (AC). Though these families share the ability to generate cAMP from ATP, they display no sequence similarity with each other. Class I are present in enteric bacteria and regulate catabolic repression, while class II are present in pathogens such as Bacillus anthracis, Pseudomonas aeruginosa and Bordetella pertusis. Classes IV, V and VI are present in Aeromonas hydrohila, Prevotella ruminicola and Rhizobium etli respectively. Class III cyclases, also known as the Universal Class, are the only class found in eukaryotes and are also present in bacteria and archaea bacteria. Mammalian cells express two different types of these enzymes; the well characterised transmembrane adenylate cyclases (tmAC) and the recently discovered soluble adenylate cyclase (solAC) (Shenoy, A. V. and Visweswariah, S. S (2004) FEBS Lett. 561, 11-21).

The tmACs are plasma membrane bound proteins. All nine of the identified tmAC isoforms (I-IX) are stimulated by hormones and neurotransmitters and activated by the α-subunit of the Gₛ protein and, with the exception of type IX, are stimulated by the diterpene forskolin. They can be differentiated by their variable responses to Gᵢ, Gₒ and G_{z} α-subunits as well as the G_{β,γ} subunits, PKC and Ca²⁺/calmodulin. It is this regulatory diversity that allows the different tmAC's to respond to intercellular signals from neurotransmitters and hormones in the correct cellular context. TmACs share a common architecture consisting of a short cytosolic N-terminus followed by a tandem repeat of a hydrophobic region, often modelled as 6 transmembrane helices, and a cytoplasmic region. The two cytoplasmic domains, termed C₁ and C₂ respectively, display a large degree of homology between themselves (˜50% identity) as well as across the nine different members of the tmAC family (˜50-90% identity). Enzymatic activity requires C₁ and C₂ to associate to form a complimentary heterodimer. Biochemical characterisation of the mechanism and regulation of these enzymes was long held up by the difficulties in purifying active proteins. A significant breakthrough came from the development of a soluble tmAC system, in which sections of the cytoplasmic domains were independently expressed as soluble recombinant proteins. Both C₁ and C₂ tend to self-associate in a head to tail interface in vitro to produce inactive (C₁) and poorly active (C₂) homodimers, but produced fully active heterodimers which retain their regulatory properties when mixed. Thus C₂ homodimers and C₁C₂ heterodimers respond to forskolin stimulation, whereas C₁ homodimers remain inactive.

The soluble tmAC systems enabled X-ray crystal studies of the enzymes, leading to the structure solution of the rat type II C₂ homodimer and the canine type V C₁/rat type II C₂ heterodimer complexed with bovine G_{sα} (Zhang, G., Liu, Y., Ruoho, A. E. and Hurley, J. H. (1997) Nature, 386, 247-253; Tesmer, J. J. G, Sunahara, R. K., Gilman, A. G. and Sprang, S. R. (1997) Science 278, 1907-1916; Tesmer, J. J. G, Sunahara, R. K., Johnson, R. A., Gosselin, G., Gilman, A. G. and Sprang, S. R (1999) Science, 285, 756-760).

The construction of such systems continues to be difficult and one of the major challenges is to determine the starting points for the C₁ and C₂ constructs. Although there is significant sequence homology across the family and construct design solely on sequence homology is a useful starting point, obtaining proteolytically stable, soluble and functional proteins is by no means routine (Beeler, J. A. and Tang, W-J. (2004) Methods in Mol. Biol, 237, 39-53).

### Soluble Adenylate Cyclase: Function, Characterisation and Purification

The existence of a new, soluble form of adenylate cyclase (“solAC”) was first reported in 1975 in the cytosolic fractions derived from rat testis. Unlike tmACs, it showed a specific requirement for Mn²⁺ ions and its activity was not stimulated by either luteinizing hormone (LH) or follicle stimulating hormone (FSH) (Braun, T. and Dods, R. F. (1975) Proc. Natl. Acad. Sci. USA, 72, 1097-1101). The ability of Mn²⁺ ions to activate the enzyme and the insensitivity the enzyme displayed to G protein regulation, enabled Buck and co-workers to biochemically and chromatographically characterise the enzyme in 1999. They identified a soluble protein with a molecular mass of 48 kDa, although the PCR isolated full length gene of solAC revealed an open reading frame encoding a much larger enzyme of 187 KDa. The 48 kD enzymatically active species was shown to reside within the N-terminal portion of the full length protein suggesting that it might represent a proteolytic fragment of the protein. Evidence for the short and full length enzyme to be generated by two alternate splice transcripts in rodent germ lines was shown by PCR and RNase protection assays (Jaiswal, B. S. and Conti, M. (2001) J. Biol. Chem., 276, 31698-31708). Comparison of the solAC open reading frame against other sequences revealed that the 48 KDa species contained two regions which displayed significant amino acid homology to various adenylate cyclase catalytic domains, the most closely related sequences being those from cyanobacteria (Anabaena spirulensis cyaB1, cyaB2 and cyaA, and Spirulina platensis cyaC) and myxobacteria (Stigmatella aurantiaca, cyaA and cyaA) (Buck, J., Sinclair, M. L. Schapal, L., Cann, M. J. and Levin, L. R. (1999) Proc. Natl. Acad. Sci. USA, 96, 79-84). Further analysis showed that the catalytic domains of solAC shared only 15 to 25% sequence identity with the tmACs' catalytic domains C₁ and C₂.

Expression of solAC has been demonstrated by reverse PCR in most tissues examined including ocular ciliary processes, corneal endothelium, choroid plexus, kidney and epididymis (Mittag, T. W., Guo, W-B and Kobayashi (1993) Am. J. Physiol 264, F1060-F1064; Zippin, J. H Levin, L. R. and Buck (2001) Trends Endocrinol Metab, 12 366-370). However Northern blot analysis and in situ hybridisation indicate, that a high degree of expression is only present in male germ cells (Sinclair, M. L., Wang, X-Y, Matia, M., Conti, M. Buck, J., Wolgemuth, D. J. and Levin, L. R. (2000) Mol. Reprod. Dev. 56, 6-11). It has been demonstrated that solAC is not solely a soluble enzyme but is specifically targeted to intracellular organelles, including mitochondria, centrioles, mitotic spindles, midbodies and nuclei, thus placing it in close proximity to effectors of cAMP signalling (Zippin, J. H., Chen, Y. Nahirney, P., Kamenetsky, M., Wuttke, M. S., Fishman, D. A., Levin, L. R and Buck, J. (2003) Faseb Journal, 17, 82-84; Litvin, T. N., Kamenetsky, M, Zarifyan, A., Buck, J. and Levin L. R. (2003) J. Biol. Chem. 278, 15922-15926).

Soluble AC is regulated by Ca²⁺ and HCO₃⁻ suggesting that modulation by these intracellular signalling molecules causes solAC to mediate the cAMP dependent responses to intrinsic cellular events. As it is the only enzyme so far identified as a bicarbonate/carbon dioxide chemosensor in mammals, solAC is thought to play a key role not only in sperm capacitation hyperactivated motility and the acrosome reaction, but also in fluid reabsorption in the kidney, fluid secretion in the ciliary bodies and choroid plexus, and metabolic regulation in response to nutritional signals (Litvin, T. N., Kamenetsky, M, Zarifyan, A., Buck, J. and Levin L. R. (2003) J. Biol. Chem. 278, 15922-15926).

The elucidation of the structure of a protein requires a source of sufficient quantities of purified protein to allow the generation of crystals. Many proteins, even when expressed in recombinant production systems, prove difficult to purify in a form in which the protein is soluble, active and non-aggregated.

A number of different bacterial adenylate cyclases have been expressed in E. coli in soluble form. In particular, the bacterial adenylate cyclases Anabaena cyaB, Mycobacterium Rv1264 and Stigmatella cyaB have been expressed in soluble form in E. coli (Cann et al. (2003), J. Biol. Chem., 278, 35033-35038; Kanacher et. Al. (2002) EMBO J. 21, 3672-3680; Linder et al. (2002) J. Biol. Chem., 277, 15271-15276).

E. coli has also been used to express Trypanosoma brucei adenylate cyclase, in the form of inclusion bodies (Bieger, B. and Essen, L-O. (2000) Acta Cryst. D56, 359-362).

E. coli has also been used to express the C2 domain of N-terminally His-tagged rat transmembrane adenylate cyclase. The protein was expressed in soluble form (Yan, S-Z et al. (1996) J. Biol. Chem., 271, 10941-10945).

The canine type V C1 domain of adenylate cyclase was also expressed, as an N-terminally His tagged fusion, by Sunahara, R. K et al. ((1997) J. Biol. Chem., 272, 22265-22271).

The elucidation of the mechanism and biochemistry of solAC has been hampered by the difficulty in obtaining sufficient material of high enough purity. The conditions for expression and recovery of the other forms of adenylate cyclase mentioned above all vary and indicate that different forms of adenylate cyclase require precise and different conditions to achieve production of substantial quantities of protein.

Prior to recombinant expression, one approach for the purification of solAC required processing of hundreds of rat testes (950) in order obtain enough protein (˜3 ug) to study the properties of the enzyme (Buck, J., Sinclair, M. L. and Levin, L. R. (2003) Methods in Enzymol. 345, 95-105). The purification procedure involved multiple steps, starting from a cell lysate comprising 20 mM Tris-HCl, pH 7.5, 1 mM DTT and protease inhibitors. Following six different column purifications, concentrated fractions of the enzyme were applied to an HPLC column and eluted with 20 mM Tris-HCl, pH 6.8, 1 mM DTT and eluted with a gradient of 0-0.1 M NaCl. It was believed that the reduction in pH—made for the first time at this stage—accounted for a significant enrichment (about 60-fold) increase in activity. The final yield of recovered solAC protein was about 3 μg, though this included a 62 KDa unrelated contaminant protein, so it is unlikely that the preparation, even if scaled up, would be suitable for the production of crystals.

Recombinant full length and catalytic domain rat solAC has also been expressed in HEK293 cells. These constructs were used to determine the properties of the enzymes encoded by the two alternate transcripts as well as the relative activities to each other and to the tissue derived equivalents. The recombinant cell lysates were clarified by centrifugation and subsequently applied to a Sephacryl S-200 column. Although yields were not reported, they were sufficient to confirm that the recombinant species corresponded in activity, size and immunoreactivity to the two solAC species expressed in the testis (Jaiswal, B. S. and Conti, M. (2001) J. Biol. Chem., 276, 31698-31708).

Recombinant approaches to expression allow the protein being expressed to be tagged to facilitate recovery. Litvin et al ((2003) J. Biol. Chem. 278, 15922-15926) describe the production of recombinant human solAC catalytic domain with a GST tagged in Baculovirus Hi 5 cells. Cells producing the protein were lysed in PBS, pH 7.4, 1 mM EDTA, 1 mM DTT and a protease inhibitor cocktail. The lysate was applied to glutathione sepharose 4B column and eluted with 50 mM Tris HCl pH 8.0, 10 mM reduced glutathione, 10 ug/ml aprotinin and 10 ug/ml leupeptin. The eluate was applied to a Superdex 200 HR 10/30 column and samples stored in 50% glycerol. The final sample contained an additional GST band and the protein of interest was not cleaved from tag. The yields were not indicated by the authors, but appear to be very low even though they were sufficient for enzymology studies.

An alternative tag is the hexahistidine tag, used by Chen et al ((2000) Science 289, 625-628) to express solAC catalytic domain (amino acids 1 to 469). The tag was fused to the C-terminus. The protein was heterologously expressed in insect HiFive cells using the Bac-to-Bac™ Baculovirus Expression system (Life Technologies) and protein was purified by chromatography over Ni2+−NTA sepharose resin (Qiagen). Conditions and yields of protein were not disclosed, yet sufficient quantities were sufficient to determine that the effect of bicarbonate on recombinant enzyme activity was not due to a pH effect. Further they showed that bisulphite ions were able to mimic the stimulation of solAC bicarbonate, whereas chloride or sulphate or phosphate did not.

Despite these previous studies with solAC, the final yields of pure recombinant mammalian protein remained low, thus preventing structural studies from being undertaken.

### SolAC Homoloque Structure

A series of structures of the solAC homolog CyaC from Spirulina platensis complexed with ATP analogs, Mg²⁺ or Ca²⁺ in the presence or absence of HCO₃⁻ have been reported. They show that the calcium ion occupies the first metal binding site and appears to mediate the binding of the nucleotide in an open conformation of the active site. The presence of bicarbonate causes the active site to close while also recruiting a second metal ion. The phosphate groups of the substrate analogs rearrange their conformation within the active site possibly to facilitate product formation and release. As the apo form of the enzyme has not been solved, it is possible that further conformations of the enzyme exist and that those observed represent trapped species along the reaction pathway (Steegborn, C., Litvin, T., Levin, L. R. Buck, J. and Wu, H. (2005) Nat. Struc. And Mol. Biol., 12, 32-37, Tesmer, J. J. G. (2005) Nat. Struc. And Mol. Biol., 12, 7-8).

### SolAC and Male Contraception

Changes in cellular cAMP concentration have been linked to the regulation of the fertilization process by spermatozoa, specifically maturation, capacitation, motility and fusion with the gamete. Despite the relevance of these events, the underlying mechanisms are poorly understood. Studies in spermatozoa to determine the specific role of the different adenylate cyclases in the modulation of these processes suggested that solAC plays a major if not the key role (de Lamirande, E., LeClerc, P. and Gagnon, C. (1997) Mol. Hum. Reprod., 3, 175-194; Jaiswal, B. J. and Conti, M. (2003) Proc. Natl. Acad. Sci. USA, 100, 10676-10681; Xie, F. and Conti, M. (2004) Developmental Biol. 265, 196-206; Chen, Y., Cann, M. J., Litvin, T. N., Iourgenko, V., Sinclair, M. L., Levin, L. R. and Buck, J. (2000) Science 289, 625-628). Definitive evidence for the role of solACs in fertilisation comes from the disruption of the solAC gene in mice causing severe impairment in sperm motility making them sterile. SolAC deficient males develop normal testes and epididymides and show no obvious abnormalities in other organs known to express solAC. Female solAC deficient mice show no phenotype and produce normal size litters with either wildtype or heterozygous mice. SolAC deficient males mated normally, yet due to the sperm's lack of forward movement failed to reproduce. In vitro studies with mutant sperm showed that normal motility could be restored by loading them with cAMP, although the mutant sperm remained unable to fertilise oocytes. (Esposito, G., Jaiswal, B. S., Xie, F., Krajnc-Franken, M. A. M., Robben, T. J. A. A, Strik, A. M., Kuil, C, Philipsen, R. L. A., van Duin, M., Conti, M. and Gossen, J. A. (2004) Proc. Natl. Acad. Sci. USA, 101, 2993-2998).

Despite the key role of solAC in fertilisation, there is evidence that several of the tmACs may also be involved in the process. Immunolocalisation of intact mouse spermatozoa showed that tmACs II, III and VIII are abundantly present in the acrosomal and flagellar regions, while tmAC I and IV are present to a lesser extent in the midpiece and acrosomal cap. As some of the components of IVF media are known to act through G-protein linked receptors, it is likely their effect is modulated via tmACs (Baxendale, R. W. and Fraser, L (2003) Mol. Reprod. and Dev. 66, 181-189).

### SolAC, Oncology, Inflammation and Other Processes

cAMP is involved in signal transduction pathways which affect cell proliferation, cell differentiation and apoptosis. Aberrations in these pathways are known to lead to pathological conditions such as cancer. Various cancers which may be mentioned include those set out in section G(vii) herein below. Thus the modulation of cAMP concentrations by increased or decreased activity of solAC can be viewed as a therapeutic or prophylactic lever.

Recent studies implicate solAC TNF-induced neutrophil activation and NGF-mediated neuritogenesis via the cAMP dependent modulation of the guanosine triphosphatase Rap-1 (Han, H., Stessin, A. M., Roberts, J., Hess, K. C., Gautam, N., Kamenetsky, M., Lou, O., Hyde, E., Nathan, N., Muller, W. A., Buck, J., Levin, L. R., and Nathan, C. (2005) J. Exp. Med., 202, 353-361; Stessin, A. M., Zippin, J. H., Kamenetsky, M., Hess, K. C., Buck, J., and Levin, L. R. (2006). J. Biol. Chem., 10, 1074). It is therefore possible that the downstream regulation of these processes, either by the activation or inhibition of solAC could be of therapeutic use.

Excessive or unregulated TNF production has been implicated in mediating or exacerbating a number of inflammatory diseases and conditions including rheumatoid arthritis (Maini et al., APMIS, 105(4): 257-263), rheumatoid spondylitis, osteoarthritis, gouty arthritis and other arthritic conditions; sepsis, septic shock, endotoxic shock, gram negative sepsis, toxic shock syndrome, adult respiratory distress syndrome, cerebral malaria, chronic pulmonary inflammatory disease, chronic obstructive pulmonary disease (COPD), acute respiratory distress syndrome (ARDS), asthma, pulmonary fibrosis and bacterial pneumonia silicosis, pulmonary sarcoisosis, bone resorption diseases, reperfusion injury, graft vs. host reaction, allograft rejections, fever and myalgias due to infection, such as influenza, herpes simplex virus type-1 (HSV-1), HSV-2, cytomegalovirus (CMV), varicella-zoster virus (VZV), Epstein-Barr virus (EBV), human herpes virus-6 (HHV-6), HHV-7, HHV-8, pseudorabies, rhinotracheitis and cachexia secondary to infection or malignancy, cachexia secondary to acquired immune deficiency syndrome (AIDS), AIDS, ARC (AIDS related complex), keloid formation, scar tissue formation, Crohn's disease, ulcerative colitis, or pyresis. Due to the inhibition of the effects caused by TNF production, it is envisaged that solAC inhibitors will be useful in the treatment of the above listed diseases.

cAMP is further known to stimulate aqueous humour formation (glaucoma) and insulin secretion from pancreatic islet cells (diabetes). Both processes are regulated by bicarbonate concentration thus linking them directly to solAC.

### Protein Crystallization

It is well known in the art of protein chemistry that crystallizing a protein is an uncertain and difficult process, without any clear expectation of success. It is now evident that protein crystallization is the main hurdle in protein structure determination. For this reason, protein crystallization has become a research subject in and of itself, and is not simply an extension of the protein crystallographer's laboratory. There are many references that describe the difficulties associated with growing protein crystals (Kierzek A M. and Zielenkiewicz P. (2001) Biophysical Chemistry, 91, 1-20 Models of protein crystal growth; Wiencek J M (1999) Annu Rev Biomed Eng., 1, 505-534 New Strategies for crystal growth; Service, R., Science (2002) November 1; 298, 948-50 Structural genomics. Tapping DNA for structures produces a trickle; Chayen N., J Struct Funct Genomics (2003) 4 (2-3) 115-20 Protein crystallization for genomics: throughput versus output).

Wiencek highlights the need for rational methodologies and protocols to produce single, high quality protein crystals suitable for protein structure determination, and mentions the lack of a fundamental approach to protein crystallization which is generally the rate-limiting step in structure determination. In connection with the need for a rational approach, the variables affecting protein solubility, nucleation and crystal growth are discussed, including the effects of temperature, pressure, pH, electrolytes, antisolvents and soluble synthetic polymers on protein solubility. Various physiochemical techniques (including laser light scattering, X-ray scattering, X-ray diffraction and atomic force microscopy) and their uses in studying crystal growth and nucleation rates are described, as are various crystal growing techniques, such as vapour diffusion, free interface diffusion, dialysis, batch growth and seeding techniques. The measurement of protein crystal quality and the variables affecting this are also discussed. Wiencek concludes that much in protein crystallization remains unclear, and that fundamental advances in our understanding of protein crystallization will have significant impact in applications beyond protein crystallography itself, because much of structural biology hinges on the ability to crystallize a protein molecule.

Similarly, Kierzek et al. acknowledge that the growth of large, well-ordered protein crystals remains the major obstacle in protein structure determination by X-ray crystallography because the physico-chemical aspect of protein crystallization is not understood. Efforts towards the formulation of models for interpreting experimental data collected thus far on protein crystal growth are reviewed, but it is stated that there are no satisfactory models of protein crystallization, because of the enormous complexity of the problem: the crystallization process spans many orders of magnitude on both time and size scales which is prohibitive for most of the computer simulation approaches. Kierzek et al. conclude that the further development of both experimental and theoretical methods will be required for some unification of the wide range of approaches currently being tested in the field of crystal growth.

Service (ibid) discusses the problems associated with crystallization and structural determination of proteins. An example given is a US pilot project initiated in order to speed up the structure determination process by automating the numerous steps involved in crystallizing proteins. This project is described as having encountered difficulties at each stage of the process: of the 1870 protein targets identified for structure determination, only 23 completed protein structures were generated. Service states that other projects have produced similar results, so that out of about 18,000 proteins targeted by various structural genomics projects worldwide, the structures of only about 200 proteins were available. The article then sets out several different points in the process of structure determination, all of which cause problems. The starting point, namely coaxing E. coli and other host cells to express the right proteins and getting them into soluble form is said to be one of the biggest problems. The continuing problem associated with getting proteins to form crystals suitable for X-ray studies is then discussed: crystallization and then optimization of the crystals is said to be a major difficulty in the process. Indeed, the article states that the attrition is severe at each step. Even if a crystal can be obtained, the structure of the crystal has to be solved, which is not a routine process, nor one where success can be expected or predicted. This is illustrated in the article by the NISGC consortium. Compared to the number of target proteins identified for crystallization and structure determination, the numbers actually cloned and then expressed as soluble protein are low. Further purification was then not always possible, and even after purification, only a small fraction of the purified proteins were crystallized. Even then, only a proportion of these proteins crystallized to produce single, high quality protein crystals suitable for protein structure determination by X-ray crystallography. Of the 5187 target proteins, Consortium members cloned 1675 of them, and expressed 1295 as protein, of which only 773 were soluble. Of these they purified 719 of their proteins, but they crystallized only 94. So far they have determined 50 structures, only 22 of which were determined by X-ray analysis.

Similar figures are presented in FIG. 1 of Chayen (ibid). Chayen focuses on the crystallization step of the many steps involved in structural genomics, and discusses the difficulty of producing high quality protein crystals suitable for structure determination by X-ray crystallography. This difficulty largely accounts for the fact that only a small percentage of the proteins produced have so far led to structure determination.

The reasons why it is commonly held that crystallization of protein molecules from solution is an obstacle in the process of determining protein structures are many; proteins are complex molecules, and the delicate balance involving specific and non-specific interactions with other protein molecules and small molecules in solution is difficult to predict.

Each protein crystallizes under a unique set of conditions, which cannot be predicted in advance. Simply supersaturating the protein to bring it out of solution will not work, the result would, in most cases, be an amorphous precipitate. Many precipitating agents are used, common ones are different salts, and polyethylene glycols, but others are known. In addition, additives such as metals and detergents can be added to modulate the behaviour of the protein in solution. Many kits are available (e.g., from Hampton Research), which attempt to cover as many parameters in crystallization space as possible, but in many cases these are just a starting point to optimize crystalline precipitates and crystals which are unsuitable for diffraction analysis. Successful crystallization is aided by knowledge of the proteins behaviour in terms of solubility, dependence on metal ions for correct folding or activity, interactions with other molecules and any other information that is available. Even so, crystallization of proteins is often regarded as a time-consuming process, whereby subsequent experiments build on observations of past trials.

In cases where protein crystals are obtained, these are not necessarily always suitable for diffraction analysis; they may be limited in resolution, and it may subsequently be difficult to improve them to the point at which they will diffract to the resolution required for analysis. Limited resolution in a crystal can be due to several factors. It may be due to intrinsic mobility of the protein within the crystal; this can be difficult to overcome, even with other crystal forms. It may be due to high solvent content within the crystal, which consequently results in weak scattering. Alternatively, it could be due to defects within the crystal lattice, which means that the diffracted x-rays will not be completely in phase from unit to unit within the lattice. Any one of these or a combination of these could mean that the crystals are not suitable for structure determination.

Some proteins never crystallize, and after a reasonable attempt it is necessary to examine the protein itself and consider whether it is possible to make individual domains, different N or C-terminal truncations, or point mutations. It is often hard to predict how a protein could be re-engineered in such a manner as to improve crystallisability. Sometimes the inclusion of a ligand in the crystallization mixture is essential for the production suitable crystals. Our understanding of crystallization mechanisms is still incomplete and the factors of protein structure, which are involved in crystallization, are not well known.

### DISCLOSURE OF THE INVENTION

The present invention relates to the crystal structure of the catalytic domain of human soluble adenylate cyclase, which allows the binding location of the substrate and co-factor in the enzyme to be investigated and determined.

Thus in one aspect, the invention provides a three dimensional apo (i.e. ligand-free) structure of soluble adenylate cyclase set out in Table 1, and uses thereof.

In a second aspect, the invention provides a three-dimensional structure of soluble adenylate cyclase in the presence of a ligand, set out in Table 2 and also in Tables 3, 4 and 5.

In general aspects, the present invention is concerned with the provision of a solAC structure and its use in modelling the interaction of ligands, e.g. potential and existing pharmaceutical compounds or other molecular structures, prodrugs, solAC modulators or substrates, or fragments of such compounds, modulators or substrates with this solAC structure.

These and other aspects and embodiments of the present invention are discussed below.

The above aspects of the invention, both singly and in combination, all contribute to features of the invention, which are advantageous.

### BRIEF DESCRIPTION OF THE DRAWINGS

FIG. 1 shows three compounds which were bound to solAC. The figure shows the atom numbering system used in Tables 3-5.

FIG. 2 shows the binding interactions of bicarbonate to three of the residues of solAC.

### BRIEF DESCRIPTION OF THE TABLES

Table 1 sets out the coordinate data of the structure of solAC.

Table 2 sets out the coordinate data of the structure of solAC in complex with AMPCPP.

Table 3 sets out the coordinate data of the structure of solAC in complex with bicarbonate

Table 4 sets out the coordinate data of the structure of solAC in complex with 5-Phenyl-2H-[1,2,4]triazole-3-thiol (Compound 1).

Table 5 sets out the coordinate data of the structure of solAC in complex with N-(3-phenoxy-phenyl)-oxalamic acid

Table 6: Active site residues of solAC.

Table 7 Active site residues of solAC interacting with adenosine moiety.

Table 8: Bicarbonate binding site of solAC.

Table 9: Channel binding site of solAC.

Table 10: Sub-pocket binding site of solAC.

Table 11: Alternative binding site residues of solAC.

Table 12: Percentage identity between entire sequences of mammalian soluble adenylate cyclases.

Table 13: Percentage identity between the catalytic domains of mammalian soluble adenylate cyclases

Table 14: solAC expression constructs.

Table 15: solAC lysis buffers.

Table 16: Heavy atom derivatives used to solve solAC structure.

### DETAILED DESCRIPTION OF THE INVENTION

### Definitions

### Selected Coordinates.

Various aspects of the invention described herein (e.g. fitting of ligands, homology modelling and structure solution, data storage means, computer assisted manipulation of the coordinates and the like), utilise the coordinates of the solAC structures set out in Table 1, Table 2, Table 3, Table 4 or Table 5, or derived from Table 1, Table 2, Table 3, Table 4 or Table 5, or obtained by reference to the coordinates of Table 1, Table 2, Table 3, Table 4 or Table 5. In all such aspects of the invention, those of skill in the art will appreciate that in many applications of the invention, it is not necessary to utilise all the coordinates of Table 1, Table 2, Table 3, Table 4 or Table 5, but merely a portion of them, e.g. a set of coordinates representing atoms of particular interest in relation to a particular use. Such a portion of coordinates is referred to herein as “selected coordinates”.

Where selected coordinates according to the invention are mentioned, it is meant for example at least 5, preferably at least 10, more preferably at least 50 and even more preferably at least 100, for example at least 500 or at least 1000 protein atoms of the solAC structure. Preferably the selected coordinates pertain to at least 30 different amino acid residues (i.e. at least one atom from 30 different residues may be present), more preferably to at least 60 residues, and even more preferably to at least 100 or 150 residues. Optionally, the selected coordinates include one or more ligand or water molecule atoms set out in Table 1, Table 2, Table 3, Table 4 or Table 5.

Adenylate cyclases in general, possess a 2-domain structure, the two domains having considerable structural homology to each other, and resembling a pseudo-dimer. Soluble adenylate cyclase is most closely related to cyanobacterial class III adenylate cyclases which function as a symmetrical homodimer. The two monomers in the homodimer, or the two domains in the pseudo-dimer may have different orientations relative to each other, depending on the contents of their active site. Therefore, the selected coordinates may be those of the N-terminal domain (residues 1-246) only or of only the C-terminal domain (residues 247-468).

In another aspect, the selected coordinates may include or may consist of atoms of one or more amino acid residues we have identified as contributing main chain or side chain atoms to the active site of solAC as described herein below, and particularly those of Table 6 (more particularly any of those of Table 7).

In a preferred embodiment, when the selected co-ordinates include at least one atom from the group of residues identified in Table 6, and more preferably from the group of residues identified in Table 7, the selected co-ordinates include at least one atom from at least 2, such as at least 3, more preferably at least 4, even more preferably at least 5 and most preferably all amino acids of these preferred groups. More preferably, the selected co-ordinates comprise at least 10, more preferably 25, more preferably 50 atoms from these groups of residues wherein at least one atom is from each member of the group.

In another aspect, the selected coordinates may include or consist of atoms of one or more amino acid residues of any one of Tables 8, 9, 10 or 11. In another embodiment, when the selected co-ordinates include at least one atom from the group of residues identified in any one of Tables 8, 9, 10 or 11, the selected co-ordinates include at least one atom from at least 2, such as at least 3, more preferably at least 4, even more preferably at least 5 and most preferably all amino acids of each table. More preferably, the selected co-ordinates comprise at least 10, more preferably 25, more preferably 50 atoms from these groups of residues wherein at least one atom is from each member of the group set out in any one of these Tables.

Alternatively, the selected coordinates may comprise at least 10, more preferably 25, more preferably 50 atoms such as at least 100 atoms from any or all of Tables 6 to 11

In one aspect, the selected coordinates may comprise one or more coordinates of an amino acid residue selected from Table 6 (e.g. a residue of Table 7) together with one or more coordinates of an amino acid residue selected from any one of Tables 8, 9, 10 or 11. In one aspect, the selected coordinates are from at least Table 6 (preferably Table 7) together with Table 8. Such groups of selected coordinates may be particularly advantageous in the design, development and analysis of ligands which occupy the ATP and bicarbonate binding sites.

In another aspect, a preferred subset of residues of Table 8 are Lys95, Val167 and Arg176. In aspects of the invention set out herein relating to the use of the coordinates of amino acids of Table 8 residues, the use of coordinates from this subset of residues is also contemplated. Additionally, the use of the coordinates of the atoms identified as having specific ligand interactions (atoms 1525, 2585, 2600, 2729 of Table 1; 1525, 2588, 2603 and 2732 of Table 2; 937, 1505, 1508 and 1578 of Table 3; 1516, 2678, 2692 and 2821 of Table 4; 1516, 2670, 2684 and 2813 of Table 5) is contemplated.

In another aspect, a preferred subset of the residues of Table 9 are His164, Phe165 and Val 335. In aspects of the invention set out herein relating to the use of the coordinates of amino acids of Table 9 residues, the use of coordinates from this subset of residues is also contemplated. Additionally, the use of the coordinates of the atoms identified as having specific ligand interactions (atoms 2536, 2546, 2565 and 5295 of Table 1; atoms 2539, 2549, 2568 and 5298 of Table 2; atoms 1482, 1486, 1489, and 2866 of Table 3; atoms 2628, 2639, 2657, and 5413 of Table 4; atoms 2620, 2631, 2649, and 5392 of Table 5) is contemplated.

In a further aspect, the selected coordinates may include or consist of atoms of one or more of the amino acid residues of an additional, partially helical domain of solAC which appears to be unique to this protein compared to tmAC. Thus the selected coordinates may include at least one atom of the residues Met1 to Tyr26 or Lys219 to Gly284. Preferably the selected coordinates include at least one atom of the residues Ile13 to His19, Phe226 to Phe236, Asp258 to Tyr268 or Glu271 to Ile277. In such a case, more preferably the selected co-ordinates include at least one atom from at least 2, such as at least 3, more preferably at least 4, even more preferably at least 5, such as at least 10 and most preferably all amino acids of these preferred coordinates. More preferably, the selected co-ordinates comprise at least 10, more preferably 25, more preferably 50 atoms from this group of residues wherein said atoms are from the preceding preferred values of different amino acids.

The selected coordinates preferably include at least about 5%, more preferably at least about 10% C-alpha atoms. Alternatively, or in addition, the selected coordinates include at least about 10%, more preferably at least about 20%, even more preferably at least about 30% backbone atoms selected from any combination of the nitrogen, C-alpha, C-terminal and carbonyl oxygen atoms.

Thus all reference to “selected coordinates” herein below should be construed as defined above unless explicitly otherwise qualified.

Root Mean Square Deviation (rmsd).

Protein structure similarity is routinely expressed and measured by the root mean square deviation (rmsd), which measures the difference in positioning in space between two sets of atoms. The rmsd measures distance between equivalent atoms after their optimal superposition. The rmsd can be calculated over all atoms, over residue backbone atoms (i.e. the nitrogen-carbon-carbon backbone atoms of the protein amino acid residues), main chain atoms only (i.e. the nitrogen-carbon-oxygen-carbon backbone atoms of the protein amino acid residues), side chain atoms only or more usually over C-alpha atoms only.

Methods of comparing protein structures are discussed in Methods of Enzymology, vol 115, pg 397-420. The necessary least-squares algebra to calculate rmsd has been given by Rossman and Argos (J. Biol. Chem., vol 250, pp 7525 (1975)) although faster methods have been described by Kabsch (Acta Crystallogr., Section A, A92, 922 (1976)); Acta Cryst. A34, 827-828 (1978)), Hendrickson (Acta Crystallogr., Section A, A35, 158 (1979)); McLachan (J. Mol. Biol., vol 128, pp 49 (1979)) and Kearsley (Acta Crystallogr., Section A, A45, 208 (1989)). Some algorithms use an iterative procedure in which the one molecule is moved relative to the other, such as that described by Ferro and Hermans (Ferro and Hermans, Acta Crystallographic, A33, 345-347 (1977)). Other methods e.g. Kabsch's algorithm locate the best fit directly.

Programs for determining rmsd include MNYFIT (part of a collection of programs called COMPOSER, Sutcliffe, M. J., Haneef, I., Carney, D. and Blundell, T. L. (1987) Protein Engineering, 1, 377-384), MAPS (Lu, G. An Approach for Multiple Alignment of Protein Structures (1998, in manuscript and on http://bioinfo1.mbfys.lu.se/TOP/maps.html)).

It is more normal when comparing significantly different sets of coordinates to calculate the rmsd value over C-alpha atoms only. However, when analysing side chain movement the rmsd over all atoms can also be calculated.

Programs such as LSQKAB (Collaborative Computational Project 4. The CCP4 Suite: Programs for Protein Crystallography, Acta Crystallographica, D50, (1994), 760-763), QUANTA (Jones et al., Acta Crystallography A47 (1991), 110-119 and commercially available from Accelrys, San Diego, Calif.), Insight (commercially available from Accelrys, San Diego, Calif.), Sybyl® (commercially available from Tripos, Inc., St Louis), O (Jones et al., Acta Crystallographica, A47, (1991), 110-119), and other coordinate fitting programs can be used to calculated rmsd values.

In, for example the programs LSQKAB and O, the user can define the residues in the two proteins that are to be paired for the purpose of the calculation. Alternatively, the pairing of residues can be determined by generating a sequence alignment of the two proteins, programs for sequence alignment are discussed in more detail in Section D. The atomic coordinates can then be superimposed according to this alignment and an rmsd value calculated. The program Sequoia (C. M. Bruns, I. Hubatsch, M. Ridderström, B. Mannervik, and J. A. Tainer (1999) Human Glutathione Transferase A4-4 Crystal Structures and Mutagenesis Reveal the Basis of High Catalytic Efficiency with Toxic Lipid Peroxidation Products, Journal of Molecular Biology 288(3): 427-439) performs the alignment of homologous protein sequences, and the superposition of homologous protein atomic coordinates. Once aligned, the rmsd can be calculated using programs detailed above. For sequence identical, or highly identical, the structural alignment of proteins can be done manually or automatically as outlined above. Another approach would be to generate a superposition of protein atomic coordinates without considering the sequence.

In various aspects of the invention described herein, the use of all or selected coordinates of Table 1, Table 2, Table 3, Table 4 or Table 5 is described. Similarly, structures and their uses obtainable by use of all or selected coordinates of Table 1, Table 2, Table 3, Table 4 or Table 5, or derived from all or selected coordinates of Table 1, Table 2, Table 3, Table 4 or Table 5, are described. In such aspects, the coordinates of the Tables may be varied within an rmsd of not more than 1.5 Å, preferably not more than 1.4 Å, more preferably not more than 1.2 Å, more preferably not more than 1.0 Å, for example preferably not more than 0.7 Å, more preferably not more than 0.5 Å, more preferably not more than 0.2 Å, and even more preferably not more than 0.1 Å.

Thus all references herein to the coordinates of Table 1, Table 2, Table 3, Table 4 or Table 5 are to be construed, unless specified to the contrary, as including an rmsd variation of not more than 1.5 Å, with preferred values of variation being that as set out in the preceding paragraph. Similarly, reference to an rmsd variation of a value smaller than not more than 1.5 Å is likewise to be construed as including the preferred, narrower, limits set out in the preceding sentence. For the avoidance of doubt, reference herein to an rmsd value of less than a specified number of Å smaller than said not more than 1.5 Å is to be understood as including the preferred, narrower, limits set out above which are not more than that specified number.

Preferably, rmsd is calculated by reference to the C-alpha atoms, provided that where selected coordinates are used, these comprise at least about 5%, preferably at least about 10%, of such atoms. Where selected coordinates do not include said at least about 5%, rmsd may be calculated by reference to all four backbone atoms, provided these comprise at least about 10%, preferably at least about 20% and more preferably at least about 30% of the selected coordinates. Where selected coordinates comprise 90% or more side chain atoms, rmsd may be calculated by reference to all the selected coordinates.

### Ligand.

As used herein, a ligand is a structure, either virtual or physical, comprising one or more atoms with a potential to bind to, or interact with, a solAC structure of the present invention. Such atoms include those found in organic molecules such as carbon, oxygen, hydrogen, nitrogen, phosphorus, and sulphur, as well as metal ions commonly found in biological systems such as iron, calcium, magnesium, manganese, selenium and the like.

A ligand for use in the present invention may be a small chemical molecule whose three-dimensional structure is available in the art, e.g. from the Cambridge Structural Database (www.ccdc.cam.ac.uk) which contains the structures of over 250,000 molecules, or may be a ligand whose structure has been designed or selected on the basis of specific structural or other criteria. These and other structures may be used for example in aspects of the invention directed to the screening of ligands in the development of new compounds which interact with solAC so as to modulate, e.g. activate or inhibit, its function. It will be apparent to those of skill in the art that whereas a ligand in the form of a virtual molecule may be made to interact with one or more protein atoms of a solAC structure of the present invention, such an interaction may not occur between the physical compound itself and solAC.

Ligands which bind to, or interact with, one or more atoms of the catalytic domain of solAC are of particular interest. A ligand may be a modulator (e.g. activator or inhibitor) of the enzyme, or a substrate for the enzyme. One such substrate may be a prodrug which is converted to an active drug by the action of the adenylate cyclase. Ligand binding is generally, though not exclusively, via non-covalent interactions, such as via hydrogen bonds or the like.

It will be apparent to those of skill in the art that reference to a ligand in the context of methods of computer-based methods of analysis and the like will refer to a virtual molecular structure, whereas in other contexts (e.g. soaking of crystals of solAC and the like) the ligand will be a chemical compound. In some aspects of the invention, a ligand will be identified by computer modelling techniques, and subsequently provided in the form of a chemical compound for further analysis. Often the analysis of the compound, e.g. in soaking or co-crystallization experiments, will lead to the production of further ligands which may then be analysed by computer-based methods of the present invention.

Candidate ligands which can be used for soaking or co-crystallization may be obtained from a variety of sources. For example, compounds under development as potential adenylate cyclase inhibitors may be used, in order to ascertain their interaction with solAC and thus to modify the ligand in a manner to enhance or otherwise modulate its activity. Such ligands may include adenine nucleotide analogues as described further below.

Alternatively, a number of synthetic compound libraries are commercially available from a various companies including Maybridge Chemical Co. (Trevillet, Cornwall, UK), Comgenex (Princeton, N.J.), Brandon Associates (Merrimack, N.H.), and Microsource (New Milford, Conn.) and a rare chemical library is available from Aldrich (Milwaukee, Wis.). Combinatorial libraries are available and can be prepared. Alternatively, libraries of natural compounds in the form of bacterial, fungal, plant and animal extracts are also available, for example, Pan Laboratories (Bothell, Wash.) or MycoSearch (N.C.), or can be readily prepared by methods well known in the art. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds.

Ligands of particular interest will be compounds under development for pharmaceutical use. Generally such ligands will be organic molecules, which are typically from about 100 to 2000 Da, more preferably from about 100 to 1000 Da in molecular weight. Such ligands include peptides and derivatives thereof, steroids, anti-inflammatory drugs, anti-cancer agents, anti-bacterial or antiviral agents, neurological agents and the like. In principle, any compound under development in the field of pharmacy can be used in the present invention in order to facilitate its development or to allow further rational drug design to improve its properties.

Other ligands of interest will be adenine nucleotides and analogues thereof. An adenine nucleotide is any one of the phosphate esters of adenosine, i.e. any one of adenosine 5′ monophosphate (AMP), ADP and ATP.

An analogue of adenine nucleotide is any compound which retains the characteristic structure of an adenine nucleotide, such as a fragment of the nucleotide or a molecular variant of the nucleotide, which is capable of binding to the ATP-binding pocket of a solAC. By characteristic structure, it is meant that the analogue will comprise one or more of a purine base structure, a sugar residue, and a mono, di- or tri-phosphate ester structure or analogue thereof.

A fragment of adenine nucleotide includes any molecular fragment of any one of the phosphate esters of adenosine, which fragment is capable of binding to the ATP-binding pocket of a solAC. Thus, examples of fragments of an adenine nucleotide are adenine (base), adenosine (nucleoside), ribose (sugar), and any one of the phosphate esters of ribose (e.g. any one of ribose 5′ monophosphate, ribose 5′ diphosphate and ribose 5′ triphosphate).

In a molecular variant of the adenine nucleotide, the purine base structure may be an adenine derivative, such as adenine substituted at position 8 (e.g. by a halogen atom to give 8-bromo ATP or 8-bromo AMP, or by an alkyl group), or in which the ring contains a heteroatom, or wherein the base is an open-ring analogue such as in ZMP (AICA riboside monophosphate). Additionally or alternatively, the sugar residue structure may comprise, for example, modifications of the 2′ or 3′ hydroxyl groups, e.g. substitution by other groups or cyclization of the groups. Furthermore, the phosphate ester portion of the analogues may be modified for example to provide for non-hydrolysable groups between the γ and β phosphates (e.g. adenosine-5′-[(β,γ)-imido]triphosphate, AMPPNP) or between the β and a phosphates (e.g. adenosine-5′-[(α,β)-methyleno]triphosphate, AMPCPP). A very large range of analogues are available from a range of commercial suppliers, e.g. Jena Bioscience GmbH (Jena, Germany).

In addition, a range of adenine nucleoside analogues are also used clinically, in particular in the antiviral area. For example vidarabine (also known as adenine arabinoside, or ara-A) is an adenine nucleoside analogue used to treat Herpes viruses by targeting viral polymerase, and 9-(3-Hydroxy-2-phosphonyl-methoxypropyl)-adenine (also known as HPMPA) is a adenine derivative with anti-HIV activity (Pauwels, R.; Balzarini, J.; Schols, D.; Baba, M.; Desmyter, J.; Rosenberg, I.; Holy, A.; De Clercq, E., Phosphonylmethoxyethyl Purine Derivatives, A New Class Of Anti-Human Immunodeficiency Virus Agents. Antimicrob Agents Chemother 32(7):1025-1030 (1988)). Some of these are phosphorylated in vivo to the triphosphate.

Adenine nucleotides and analogues thereof may be used as ligands which can be modified further to increase or decrease their interactions with solAC, e.g. in the development of novel pharmaceutical compounds.

Ligands may also be compounds with a moiety which is a bicarbonate or bisulphide analogue moiety, such that the moiety has the ability to coordinate one or more of the solAC atoms involved in bicarbonate binding.

### A. Protein Crystals.

The present invention provides a crystal of the human soluble adenylate cyclase catalytic domain.

In a further aspect, the invention provides a crystal of the soluble adenylate cyclase catalytic domain comprising residues 1 to 468 of SEQ ID NO:3 or a variant thereof having from 1 to 10 amino acid substitutions, deletions or insertions. Such a crystal may have the sequence of SEQ ID NO:3, optionally excluding the His6 tag (i.e. 1 to 469 of SEQ ID NO:3) or wherein the His6 tag is replaced by a tag having from 4 to 20 amino acids. Such a tag may comprise a smaller or larger number of histidine residues, e.g. may be a His4, His5, His7 or His8 tag. The crystal may comprise an allele or a variant of residues 1-468 of SEQ ID NO:3 which retains the ability to form crystals under the conditions illustrated herein. Such variants include those with a number of amino acid substitutions, for example 1, 2, 3, 4, 5, 6, 7, 8, 9 or 10 amino acids by an equivalent or fewer number of amino acids. Further examples of variants, including mutants, are discussed further herein below.

In one embodiment, the crystals of the invention described above have a space group P6₃ with unit cell dimensions a=b=99.5 Å, c=97.4 Å, and α=β=90.0°, γ=120.0°. Unit cell dimensions may be subject to a variability of 5%. Thus, particular examples are a=b=99.51 Å, c=97.13 Å; a=b=99.424 Å, c=97.390 Å (as shown in Table 1); a=b=99.651 Å, c=96.522 (as shown in Table 2); a=b=99.673 Å, c=96.824 (as shown in Table 3); a=b=99.569 Å, c=98.470 (as shown in Table 4); a=b=99.291 Å, c=97.753 (as shown in Table 5).

More generally, the unit cell crystal dimensions may be in the ranges 104.475 Å>a=b>94.525 Å, 102.27 Å>c>92.53 Å.

Such crystals may be obtained using the methods described in the accompanying examples.

The invention provides a crystal of the solAC catalytic domain grown in the absence of any active site binding ligand. The invention therefore further includes the apo crystal of the solAC catalytic domain.

The methodology used to provide a solAC crystal or co-crystal illustrated herein may be used generally to provide a solAC catalytic domain crystal or co-crystal resolvable at a resolution of about 3.5 Å or better.

The invention thus further provides a solAC catalytic domain crystal or co-crystal having a resolution of 3.5 Å or better.

In a further aspect, the invention provides a method for making a solAC catalytic domain protein crystal, particularly of a solAC protein comprising the sequence of the catalytic domain of solAC or a variant thereof, which method comprises growing a crystal by hanging drop vapour diffusion.

A further aspect of the invention provides a solAC catalytic domain into which a ligand has been soaked

Further, the invention provides a method for making a crystal of a complex of solAC catalytic domain with a ligand, which method comprises taking a crystal of solAC catalytic domain and soaking a ligand into it.

### (i) Mutants

A mutant is a solAC catalytic domain protein characterized by the replacement, insertion or deletion of at least one amino acid from the wild type solAC. Such a mutant may be prepared for example by site-specific mutagenesis, or incorporation of natural or unnatural amino acids.

The present invention contemplates “mutants” wherein a “mutant” refers to a polypeptide which is obtained by replacing at least one amino acid residue in a native or synthetic solAC with a different amino acid residue and/or by adding and/or deleting amino acid residues within the native polypeptide or at the N- and/or C-terminus of a polypeptide corresponding to solAC, and which has substantially the same three-dimensional structure as solAC from which it is derived. By having substantially the same three-dimensional structure is meant having a set of atomic structure co-ordinates that have a root mean square deviation (rmsd) of less than about 1.5 Å from the coordinates of Table 1, Table 2, Table 3, Table 4 or Table 5.

A mutant may have, but need not have, enzymatic or catalytic activity.

To produce homologues or mutants, amino acids present in the said protein can be replaced by other amino acids having similar properties, for example hydrophobicity, hydrophobic moment, antigenicity, propensity to form or break α-helical or β-sheet structures, and so on. Substitutional variants of a protein are those in which at least one amino acid in the protein sequence has been removed and a different residue inserted in its place. Amino acid substitutions are typically of single residues but may be clustered depending on functional constraints e.g. at a crystal contact. Preferably amino acid substitutions will comprise conservative amino acid substitutions. Insertional amino acid variants are those in which one or more amino acids are introduced. This can be amino-terminal and/or carboxy-terminal fusion as well as intrasequence. Examples of amino-terminal and/or carboxy-terminal fusions are affinity tags (e.g. MBP or GST tags), or epitope tags.

Amino acid substitutions, deletions and additions which do not significantly interfere with the three-dimensional structure of the solAC will depend, in part, on the region of the solAC where the substitution, addition or deletion occurs. In highly variable regions of the molecule, non-conservative substitutions as well as conservative substitutions may be tolerated without significantly disrupting the three-dimensional structure of the molecule. In highly conserved regions, or regions containing significant secondary structure, conservative amino acid substitutions are preferred.

Conservative amino acid substitutions are well-known in the art, and include substitutions made on the basis of similarity in polarity, charge, solubility, hydrophobicity, hydrophilicity and/or the amphipathic nature of the amino acid residues involved. For example, negatively charged amino acids include aspartic acid and glutamic acid; positively charged amino acids include lysine and arginine; amino acids with uncharged polar head groups having similar hydrophilicity values include the following: leucine, isoleucine, valine; glycine, alanine; asparagine, glutamine; serine, threonine; phenylalanine, tyrosine. Other conservative amino acid substitutions are well known in the art.

In some instances, it may be particularly advantageous or convenient to substitute, delete and/or add amino acid residues in order to provide convenient cloning sites in the cDNA encoding the polypeptide, to aid in purification of the polypeptide, etc. Such substitutions, deletions and/or additions which do not substantially alter the three dimensional structure of solAC will be apparent to those having skills in the art.

As mentioned above, the mutants contemplated herein need not exhibit enzymatic activity. Indeed, amino acid substitutions, additions or deletions that interfere with the catalytic activity of the solAC but which do not significantly alter the three-dimensional structure of the catalytic region are specifically contemplated by the invention. Such crystalline polypeptides, or the atomic structure co-ordinates obtained there from, can be used to identify ligands that bind to the protein.

The residues for mutation could easily be identified by those skilled in the art and these mutations can be introduced by site-directed mutagenesis e.g. using a Stratagene QuikChange™ Site-Directed Mutagenesis Kit or cassette mutagenesis methods (see e.g. Ausubel et al., eds., Current Protocols in Molecular Biology, John Wiley & Sons, Inc., New York, and Sambrook et al., Molecular Cloning: a Laboratory Manual, 2nd ed., Cold Spring Harbor Laboratory Press, Cold Spring Harbor, N.Y., (1989)).

### (ii) Alleles

The present invention contemplates “alleles” wherein allele is a term coined by Bateson and Saunders in 1902 for characters which are alternative to one another in Mendelian inheritance. Now the term allele is used for two or more alternative forms of a gene resulting in different gene products and thus different phenotypes. An allele contains nucleotide changes that have been shown to affect transcription, splicing, translation, post-transcriptional or post-translational modifications or result in at least one amino acid change. Typically, an allelic variant of a solAC will have at least 75% sequence identity (more preferably, at least 80%, 85%, 90% or 95% sequence identity) with the corresponding specifically exemplified solAC protein, where sequence identity is determined by comparing the nucleotide sequences of the polynucleotides when aligned so as to maximize overlap and identity while minimizing sequence gaps. More usually allelic forms comprise 1-10 amino acid changes, in particular 1 or 2 amino acid changes, from the wild-type protein, or 1-30 nucleotide changes in the DNA sequence.

To the extent that the present invention relates to solAC-ligand complexes and mutant, homologue, analogue, allelic form, species variant proteins of solAC, crystals of such proteins may be formed. The skilled person would recognize that the conditions provided herein for crystallising solAC may be used to form such crystals. Alternatively, the skilled person would use the conditions as a basis for identifying modified conditions for forming the crystals.

Thus the aspects of the invention relating to crystals of solAC, may be extended to crystals of mutant and mutants of solAC which result in homologue, allelic form, and species variant.

(iii) Purification of solAC

To obtain sufficient quantity of protein for crystallization, it was necessary to develop a protocol for high level expression and recovery of solAC. In one embodiment, the invention provides a process for the production of solAC (for example the solAC comprising residues 1-469 of SEQ ID NO:3 or a mutant or variant thereof), the solAC optionally being fused to a C-terminal or N-terminal tag, which method comprises
expressing the solAC in a eukaryotic cell culture;
lysing the cells of the culture in a buffer comprising 10-100 mM Tris pH7.4-8.0 (at 4° C.), 0.3-0.5 M NaCl, 0-20% (v/v) glycerol and 2-5 mM beta-mercaptoethanol; and
recovering the solAC from the culture.

More preferably, the process comprises:
lysing the cells of the culture in a buffer comprising 40-60 mM Tris pH7.4-7.6. (at 4° C.), 0.3-0.4 M NaCl, 5-15% (v/v) glycerol and 2-5 mM beta-mercaptoethanol; and
recovering the solAC from the culture.

Even more preferably, following expression in said cell culture, the process comprises:
lysing the cells of the culture in a buffer comprising 50 mM Tris pH7.5 (at 4° C.), 0.3 M NaCl, 10% (v/v) glycerol and 2-5 mM beta-mercaptoethanol; and
recovering the solAC from the culture.

In these above-mentioned processes, the eukaryotic cell culture may be a mammalian or insect cell culture, particularly an insect cell culture.

In one embodiment, the solAC comprises a histidine tag (e.g. a tag comprising from about 4 to 10, such as about 6 histidine) residues and the recovery of the solAC includes the step of binding the solAC to a chelate column under conditions for binding of the tag to the column, followed by elution of the protein from the column. In another embodiment, the solAC may comprise a GST tag.

The invention also provides a composition comprising solAC—particularly the solAC of SEQ ID NO:3 or a mutant or variant thereof—in a buffer comprising 50 mM Tris pH7.5 (at 4° C.), 330 mM NaCl, 10% (v/v) glycerol and 1 mM beta-mercaptoethanol. In a preferred embodiment, the solAC is at a concentration of from 10 to 40 mg/ml, such as from 20 to 40 mg/ml. Preferably, the solAC remains in monomeric form in these preferred concentrations.

(iv) Crystallization of solAC

To produce crystals of solAC catalytic domain protein the final protein is concentrated to ˜8-15 mg/ml in a buffer, for example comprising 50 mM Tris, pH7.5, 330 mM sodium chloride, 1 mM beta-mercaptoethanol and 10% glycerol, by using concentration devices which are commercially available.

Crystallization of the protein is set up by the hanging or sitting drop methods and the protein is crystallized by vapour diffusion at 4° C. against a range of vapour diffusion buffer compositions. Microseeding may be used. It is customary to use a 1:1 ratio of protein solution and vapour diffusion buffer in the hanging or sitting drop, and this has been used herein unless stated to the contrary. The hanging or sitting drop typically has a volume of from about 0.5 to 2 μl, such as about 1 to 2 μl, preferably 1 μl. Crystallization may also be performed by the microbatch method.

Typically, the vapour diffusion buffer comprises 100 mM sodium acetate pH4.8, 200 mM trisodium citrate, 14-17% PEG4000, 10% glycerol and 3 mM beta-mercaptoethanol. Trial conditions for crystallization may be prepared using a Hampton Research Screening kits, Poly-ethylene glycol (PEG)/ion screens, PEG grid, Ammonium sulphate grid, PEG/ammonium sulphate grid or the like.

Additives can be added to a crystallization condition identified to influence crystallization. Additive Screens are to be used during the optimisation of preliminary crystallization conditions where the presence of additives may assist in the crystallization of the sample and the additives may improve the quality of the crystal e.g. Hampton Research additive screens which use glycerol, polyols and other protein stabilizing agents in protein crystallization (R. Sousa. Acta. Cryst. (1995) D51, 271-277) or divalent cations (Trakhanov, S, and Quiocho, F. A. Protein Science (1995) 4,9, 1914-1919).

In addition, detergents may be added to a crystallization condition to improve the crystallization behaviour e.g. the ionic, non-ionic and zwitterionic detergents found in the Hampton Research detergent screens (McPherson, A., et al., The effects of neutral detergents on the crystallization of soluble proteins, J. Crystal Growth (1986) 76, 547-553).

In addition crystal quality may be improved by the use of seeding methods. These include microseeding, streak seeding and macroseeding. A commercial seed preparation kit may be used, such as those sold by Hampton Research.

### B. Crystal Coordinates.

In a further aspect, the invention also provides a crystal of soluble adenylate cyclase catalytic domain having the three dimensional atomic coordinates of Table 1.

Table 1 gives atomic coordinate data for the catalytic domain of soluble adenylate cyclase present in the asymmetric unit, identified as molecule A encompassing atoms 1 to 7273. In these rows of the Table the second column denotes the atom number, third column denotes the atom, the fourth the residue type, the fifth the chain identification (A or B), the sixth the residue number the seventh, eighth and ninth columns are the X, Y, Z coordinates respectively of the atom in question, the tenth column the occupancy of the atom, the eleventh the temperature factor of the atom, the twelfth the atom type.

The remaining atoms of the Table (7274-8726) are water, with the atoms identified in the same format as above.

An advantageous feature of the structures defined by the atomic coordinates of Table 1 are that they have a resolution of about 1.7 Å. An advantageous feature of the structures defined by the atomic coordinates of Table 2 are that they have a resolution of about 1.9 Å. An advantageous feature of the structures defined by the atomic coordinates of Table 3 and Table 4 are that they have a resolution of about 2.0 Å. An advantageous feature of the structures defined by the atomic coordinates of Table 5 are that they have a resolution of about 2.05 Å.

A particular advantage of the structure as defined by the atomic coordinates of Table 1 are that they delineate a structure having an active site which is unoccupied by ligand.

In a further aspect, the invention also provides a crystal of soluble adenylate cyclase catalytic domain in complex with the molecule AMPCPP, having the three-dimensional structure set out in Table 2.

Table 2 gives atomic coordinates for the complex of solAC with AMPCPP, where the protein identified as molecule A comprises atoms numbered from 1-7289, the AMPCPP molecule comprises atoms numbered 7291-7339, a calcium ion is numbered 7340 and the remaining atoms are numbered 7341-8307 and are water molecules. In these rows of the Table the second column denotes the atom number, the third column denotes the atom, the fourth the residue type, the fifth the chain identification (A or B), the sixth the residue number the seventh, eighth and ninth columns are the X, Y, Z coordinates respectively of the atom in question, the tenth column the occupancy of the atom, the eleventh the temperature factor of the atom, the twelfth the atom type.

Table 3 gives the atomic coordinate data for the complex of solAC with bicarbonate. The protein identified as molecule A comprises atoms numbered from 171 to 3909. These atoms are preceded in the table by the water molecules (1-87 and 92-170) and atoms of bicarbonate (88-91).

Table 4 gives atomic coordinate data for the complex of solAC with 5-Phenyl-2H-[1,2,4]-triazole-3-thiol (compound 1), wherein the protein identified as molecule A comprises atoms numbered from 1-7499, the compound 1 molecule is atoms 7516-7534 and the molecules after this are water. There is also a glycerol molecule present in the structure as atoms 7502 to 7514.

Table 5 gives atomic coordinate data for the complex of solAC with N-(3-phenoxy-phenyl)-oxalamic acid (compound 2), wherein the protein identified as molecule A comprises atoms numbered from 1-7478, the compound 2 molecule is atoms 7495-7523 and the molecules after this are water. There is also a glycerol molecule present in the structure as atoms 7481 to 7493.

The order of the columns in Tables 3-5 are as for Table 2 in relation to the solAC protein structure.

Table 1, Table 2, Table 3, Table 4 and Table 5 are all set out in internally consistent formats. For example in Tables 1, 2, 4 and 5 the coordinates of the atoms of each amino acid residue are listed such that the backbone nitrogen atom is first, followed by the C-alpha backbone carbon atom, designated CA, followed by side chain residues atoms (designated according to one standard convention) and finally by the carbon and oxygen of the protein backbone. In Table 3 the carbon and oxygen of the protein backbone precede the side chain residues. Table 3 also contains consecutive atom numbering for the solAC structure, whereas the numbering of the other tables uses the convention of counting the hydrogen atoms of the protein molecule. Alternative file formats which may include a different ordering of these atoms, or a different designation of the side-chain residues, ligand molecule atoms, may be used or preferred by others of skill in the art. However it will be apparent that the use of a different file format to present or manipulate the coordinates of the Table is within the scope of the present invention.

The coordinates of Table 1, Table 2, Table 3, Table 4 or Table 5 provide a measure of atomic location in Angstroms, given to 3 decimal places. The coordinates are a relative set of positions that define a shape in three dimensions, but the skilled person would understand that an entirely different set of coordinates having a different origin and/or axes could define a similar or identical shape. Furthermore, as set out in the “Definitions” section above, the skilled person would understand that varying the relative atomic positions of the atoms of the structure so that the root mean square deviation is less than 1.5 Å will generally result in a structure which is substantially the same as the structure of Table 1, Table 2, Table 3, Table 4 or Table 5 in terms of both its structural characteristics and usefulness for structure-based analysis of solAC-interactivity with ligands.

Likewise the skilled person would understand that changing the number and/or positions of the water molecules and/or ligand molecules of the Tables will not generally affect the usefulness of the structures for structure-based analysis of solAC-interacting ligands. Thus for the purposes described herein as being aspects of the present invention, it is within the scope of the invention if: the coordinates of Table 1, Table 2, Table 3, Table 4 or Table 5 are transposed to a different origin and/or axes; the relative atomic positions of the atoms of the structure or selected coordinates thereof are varied so that the root mean square deviation of the resulting varied structure is less than 1.5 Å when superimposed on the coordinates provided in Table 1, Table 2, Table 3, Table 4 or Table 5; and/or the number and/or positions of water molecules and/or ligand molecules is varied.

As mentioned above, preferred selected coordinates of the solAC structures of Table 1, Table 2, Table 3, Table 4 or Table 5 may be atoms of one or more amino acid residues we have identified as contributing main chain or side chain atoms to the active site of solAC as described herein below. Such atoms include one or more of those present in the amino acids set out in any one of Tables 6, 7, 8, 9, 10 or 11 such as Tables 6, 7 or 11. Preferred selected coordinates of each Table, or combinations of coordinates from two or more Tables are as described elsewhere herein.

The structure of the catalytic domain of soluble adenylate cyclase is described by the coordinates in Table 1 or Table 2. Residue Met1 is the first observable residue in the electron density and Lys468 is the last. An electron density peak close to the main chain nitrogen of Met1 may be due to acetylation, and has not been modelled. Residues which have no interpretable main chain density in Table 1 are Trp135, Glu136, Glu137, Gly138, Leu139, Asp140, Phe350, Pro351, Gly352, Glu353, Lys354, Val355 and Pro356. Residue Val469 and the C-terminal His tag are not visible in the electron density. The main chain close to the first break at 134 is poorly defined and residues 132-134 have patchy main chain electron density. Residues 304-306 and 451-455 are poorly defined by the electron density. In addition, several residues on the periphery of the model have had their side-chains placed arbitrarily as there is no interpretable density for them. Phe350 is visible in Table 2.

Further structures of the catalytic domain of soluble adenylate cyclase were resolved and several of these structures had interpretable density for the unresolved residues of Table 1, allowing the more complete coordinate data of Table 3, Table 4 or Table 5 to be obtained.

In order to compare the solved mammalian soluble adenylate cyclase structure with the previously published structure of bacterial soluble adenylate cyclase, the rmsd between the soluble adenylate cyclase and the Spirulina platensis adenylate cyclase (PDB code 1wc1) (Steegborn, C., Litvin, T., Levin, L. R. Buck, J. and Wu, H. (2005) Nat. Struc. And Mol. Biol., 12, 32-37) structures was calculated. Calculations were carried out using Comparer (Sali and Blundell (1990) J Mol. Biol. 212, 403-428) to superpose the structures and MNYFIT (Sutcliffe et al. 1987) to refine the superposition and calculate rmsd. Rmsd was calculated using positions of the Cα atoms only. Each structure was split into its constituent domains in order to perform the superposition. This negates the issue of domain movement being the cause of large differences in rmsd between the structures. The first domain of the soluble adenylate cyclase (residues 36-216) was therefore superimposed on the B chain of the Spirulina platensis structure and the second domain (residues 287-465) on the C chain. A cut-off of 2.5 Å was used to define equivalence (Cα atoms of one structure that are within 2.5 Å of Cα atoms of the superposed structure are equivalent). For the first domain of the soluble adenylate cyclase 116 of the 181 residues were defined as equivalent to residues in the S. platensis structure with an rmsd of 1.2 Å. For the second domain, 127 of the 179 residues are defined as equivalent to residues in the S. platensis structure with an rmsd of 1.4 Å. These rmsd values and the low number of equivalent residues identified at the 2.5 Å cut-off are a consequence of the difference in length and orientation of a number of the regions of sequence joining secondary structure elements. Inspection of the superposition also reveals a difference in the length of helix α3, which is several residues shorter in the mammalian cyclase and while the core β-sheet portion of the molecules superpose well, helices α2 and α3 superpose less well. Thus, overall the two domains of the solAC structure of the present invention have rmsd values of total Cα atoms of considerably more than 1.5 Å from the S. platensis structure 1wc1.

There are some differences in secondary structure between the enzymes, e.g. human soluble adenylate cyclase has a shorter beta1 strand and a longer alpha1 helix than the tmAC or the S. platensis enzyme.

A major difference between solAC and both the tmACs and the S. platensis solAC is the presence of an additional partially helical domain lying against the base of the N-terminal domain of the pseudodimer. It consists of N-terminal residues 1-26, residues 1-12 have an extended conformation, 13-19 adopt a helical conformation, the remaining residues 20-26 extended with 25-27 forming one turn of a helix. The remainder of the domain is formed by residues 219-284. This includes residues which would form beta8 in the N-terminal domain, beta1 in the C-terminal domain, and alpha1 in the C-terminal domain. Within residues 219-284, the residues 226-236, 258-268 and 271-277 form three helices.

We have identified several regions within solAC which bind ligands. We have also found that these regions have the potential to form an expanded binding site for ligands which bind in two or more of these regions.

The regions we have identified are (a) the ATP binding site, (b) the bicarbonate binding site, (c) a channel binding site and (d) a sub-pocket binding site. Further, we have also identified (e) an alternative, potentially allosteric, site. A more detailed description of these sites is provided in the accompanying examples.

### (A) The ATP Binding Site.

This site, referred to more generally as the active site of solAC, is lined by active site residues. The active site residues of solAC identified by the present invention are set out in single letter code in Tables 6 and 7 which follow.

TABLE 6
Active site residues of solAC

| | | | |
|---|---|---|---|
| D47 | I48 | S49 | G50 |
| F51 | T52 | A97 | G98 |
| F296 | F336 | F338 | L345 |
| V406 | V411 | N412 | A415 |
| R416 | | | |

TABLE 7
Active site residues of solAC interacting with adenosine moiety.
Residues forming hydrogen bonds with the adenine group are
differentiated from those which line the sides of the hydrophobic pocket.

| | | | | | | | |
|---|---|---|---|---|---|---|---|
| H-bonds | G98 | V406 | | | | | |
| Hydrophobic | A97 | F296 | F336 | F338 | L345 | V411 | A415 |
| pocket | | | | | | | |

### (B) Bicarbonate Binding Site

Our analysis of the structure of solAC has revealed a bicarbonate binding region. Three residues of solAC interact with a bicarbonate ion, namely Lys95, Val167 and Arg176. Using a compound (5-Phenyl-2H-[1,2,4]-triazole-3-thiol; “compound 1”) with a negatively charged sulphur atom that sits in an almost identical position to the HCO₃⁻ ion we have identified an expanded bicarbonate binding site which forms a large pocket, contiguous with the ATP binding site.

This expanded HCO₃⁻ binding site highlights a region of the solAC structure that can be exploited in the design of solAC inhibitors or activators.

The residues of the expanded site are as set out in Table 8.

TABLE 8
Bicarbonate binding site residues.

| | | | |
|---|---|---|---|
| Phe45 | Leu94 | Lys95 | Ala97 |
| Ala100 | Leu102 | Phe165 | Leu166 |
| Val167 | Ile168 | Val172 | Arg176 |
| Val335 | Phe336 | Met337 | Phe338 |

In these residues, we have found that:
1) The NZ of Lys95 forms a salt bridge with the O1 of HCO₃⁻;
2) the backbone NH of Val167 forms a charged hydrogen bond with the O1 of HCO₃;
3) the backbone carbonyl of Val167 forms a hydrogen bond with the OH of HCO₃⁻;
4) the side-chain NH2 of Arg176 forms a salt bridge with the O3 of HCO₃⁻ and
5) the side-chain NH2 of Arg176 forms a charged hydrogen bond with the OH of HCO₃⁻.

Within the extended bicarbonate site, the sulphur atom of 5-Phenyl-2H-[1,2,4]-triazole-3-thiol is located at the bottom of the pocket and forms a salt bridge with the NZ of Lys95 and charged hydrogen bonds with the backbone NH of Val167 and the backbone NH of Phe336. The N6 of the triazole also forms a hydrogen bond with the backbone carbonyl of Met337 at the bottom of the pocket. In addition, the N5 of the triazole forms a charged hydrogen bond with the NH2 of Arg176. The phenyl group of compound 1 extends into a predominantly lipophilic region of the compound pocket formed by the sidechains of Phe45, Lys95, Ala97, Ala100, Leu102 and Phe336. Beyond the phenyl group of the compound the pocket narrows slightly before opening into the ATP binding site. The shape of the expanded HCO₃⁻ binding site and the nature of the interactions observed in the compound:solAC complex suggest that this site will be amenable to a variety of ligands.

### (C) Channel Binding Site

The crystal structure of N-(3-phenoxy-phenyl)-oxalamic acid (“compound 2”) bound to solAC reveals further regions of the solAC structure that might be exploited in drug design. The binding site for compound 2 overlaps with the expanded HCO₃⁻ pocket described for compound 1 such that the phenoxy group of compound 2 occupies a very similar position to the phenyl group of compound 1. However, compound 2 induces a large sidechain movement of Lys95, which lies at the bottom of the HCO₃⁻ binding site. This Lys95 movement opens up the HCO₃⁻ binding pocket to form a channel that merges with a large water filled cavity before opening onto the protein surface at a point opposite to the ATP binding site. The following residues (Table 9) line this newly exposed channel:

TABLE 9
Channel Binding Site

| | | |
|---|---|---|
| His164 | Phe165 | Tyr268 |
| Asn333 | Lys334 | Val335 |

The oxalamic acid group of compound 2 protrudes furthest into this channel to form several interactions with the protein:

1) the compound 2 O3 forms a charged hydrogen bond with the ND of His164;
2) the compound 2O1 forms a charged hydrogen bond with the backbone NH of Phe165,
3) the compound 2O5 hydrogen bonds to the backbone NH of Val335 and
4) the amide N6 of compound 2 hydrogen bonds to the backbone carbonyl of Phe165.

The side-chains of Lys95, Phe165, Leu166, Val167 and Phe336 form a lipophilic environment around the central anilino aromatic ring of compound 2. Although the terminal phenoxy group of compound 2 binds within the same lipophilic pocket described for compound 1, the environment of this pocket is slightly altered via a compound 2 induced movement of a loop comprising Met337, Phe338, Asp339, Lys340 and Gly341. This loop movement drags Phe338 away from the ATP site to within van der Waals distance of the terminal phenoxy group of compound 2. This movement of Phe338 is not observed in the structures of apo, AMP-PNP and compound 1 bound solAC.

### (D) Sub-Pocket Binding Site

Further analysis of the structure of the AMP-PNP complex reveals that Phe338 forms one end of the ATP site sitting close to the hydroxyl groups of the AMP-PNP ribose. The repositioning of Phe338 creates a new sub-pocket adjacent to the ATP binding site. This new sub-pocket is lined by the following residues (Table 10)

TABLE 10
Sub-pocket residues:

| | | | |
|---|---|---|---|
| Phe296 | Met418 | Asn298 | Ser343 |
| Phe336 | Met337 | Gly341 | Asp339 |
| Met419 | Cys342 | Ala415 | Arg416 |
| Met300 | Lys340 | | |

(E) Alternative Binding Site on the solAC

A hydrophobic cleft on the surface of the solAC molecule forms a binding site for the N-terminus of a symmetry related molecule The side chain of the N-terminal methionine slots into a hydrophobic pocket formed by the side-chains of Phe89, Phe230 and Phe226. The main chain of residues 1-4 then slot into a groove formed on one side by residues at the end of helix 2 and on the other side by residues from the loop including residue numbers Lys246, Asn247, Leu248 and Leu249, which lie in an extra domain of solAC relative to Spirulina platensis adenylate cyclase. While not wishing to be bound by any one particular theory, these residues, set out in Table 11, may form an allosteric site and thus this may be a further target for ligands to solAC.

TABLE 11
Alternative binding site residues

| | | | |
|---|---|---|---|
| Phe89 | Phe226 | Phe230 | Lys246 |
| Asn247 | Leu248 | Leu249 | |

### Complex Structures

The structure of the soluble adenylate cyclase soaked with AMPCPP provides structural information on the nucleotide binding pocket of the protein, in particular that of the adenosine moiety of the nucleotide. This structure may then be used to model interactions of analogous structures with solAC, for the purpose of designing more potent modulators, e.g. inhibitors or activators. More generally, the structure of soluble adenylate cyclase soaked with other ligands provides structural information on the other binding pockets of this protein.

Identification and Use of solAC Residues.

The crystal structure for solAC has allowed the precise identification of all the residues that line the binding site of the enzyme (Table 6) in the ATP binding pocket region.

Accordingly, in a preferred aspect of the invention, where the invention contemplates the use of selected coordinates in a method of the invention, such selected coordinates will comprise at least one coordinate, preferably at least one side-chain coordinate, of an amino acid residue selected from Table 6. More preferably, the selected coordinates comprise at least one coordinate, preferably at least one side-chain coordinate, of an amino acid residue selected from Table 7. The residues identified in Table 6 are particularly useful for small molecule ligand design.

Also preferred, whether all or just some atoms of a particular amino acid are selected, is that at least 5, more preferably at least 10, and most preferably at least 50 of the selected coordinates are of side chain residues from the corresponding number of different amino acid residues. These may be selected exclusively from Table 6, 7 or 11.

In other embodiments, where the invention contemplates the use of selected coordinates in a method of the invention, such selected coordinates will comprise at least one coordinate, preferably at least one side-chain coordinate, of an amino acid residue selected from any one of Table 6, 7 or 11.

### C. Chimeras.

The use of chimeric proteins to achieve desired properties is now common in the scientific literature. Thus, a variant of solAC may be a chimera.

Of particular relevance are cases where the active site is modified so as to provide a surrogate system to obtain structural information. For example, Ikuta et al (J Biol Chem (2001) 276, 27548-27554) modified the active site of cdk2, for which they could obtain structural data, to resemble that of cdk4, for which no X-ray structure is currently available. In this way they were able to obtain protein/ligand structures from the chimaeric protein which were useful in cdk4 inhibitor design. In a similar way, based on comparison of primary sequences of related solAC isoforms, the binding site of the solAC of the present invention could be modified to resemble those isoforms. Protein structures or protein/ligand structures of the chimaeric proteins could be used in structure-based selection of compounds which are ligands of that related solAC isoform.

Even if the percentage of the amino acid sequence identity between solAC described herein and other isoforms rank from 20 to 80%, the overall folding of solAC proteins is expected to be very similar, with the same spatial distribution of the structural elements.

### D. Homology Modelling.

The invention also provides a means for homology modelling of other proteins (referred to below as target adenylate cyclase proteins). By “homology modelling”, it is meant the prediction of related adenylate cyclase structures using computer-assisted de novo prediction of structure, based upon manipulation of the coordinate data derivable from Table 1, Table 2, Table 3, Table 4 or Table 5, or selected coordinates thereof.

“Homology modelling” extends to target adenylate cyclase proteins which are analogues or homologues of the solAC protein whose structure has been determined in the accompanying examples. It also extends to protein mutants of solAC protein itself.

The term “homologous regions” describes amino acid residues in two sequences that are identical or have similar (e.g. aliphatic, aromatic, polar, negatively charged, or positively charged) side-chain chemical groups. Identical and similar residues in homologous regions are sometimes described as being respectively “invariant” and “conserved” by those skilled in the art.

In general, the method involves comparing the amino acid sequences of the solAC protein with a target protein by aligning the amino acid sequences. Amino acids in the sequences are then compared and groups of amino acids that are homologous (conveniently referred to as “corresponding regions”) are grouped together. This method detects conserved regions of the polypeptides and accounts for amino acid insertions or deletions.

Homology between amino acid sequences can be determined using commercially available algorithms. The programs BLAST, gapped BLAST, BLASTN, PSI-BLAST, BLAST2 and WU-BLAST (provided by the National Center for Biotechnology Information) are widely used in the art for this purpose, and can align homologous regions of two, or more, amino acid sequences. These may be used with default parameters to determine the degree of homology between the amino acid sequence of the solAC and other target proteins which are to be modelled.

Preferred for use according to the present invention is the WU-BLAST (Washington University BLAST) version 2.0 software. WU-BLAST version 2.0 executable programs for several UNIX platforms can be downloaded from ftp://blast.wustl.edu/blast/executables. This program is based on WU-BLAST version 1.4, which in turn is based on the public domain NCBI-BLAST version 1.4 (Altschul and Gish, 1996, Local alignment statistics, Doolittle ed., Methods in Enzymology 266: 460-480; Altschul et al., 1990, Basic local alignment search tool, Journal of Molecular Biology 215: 403-410; Gish and States, 1993, Identification of protein coding regions by database similarity search, Nature Genetics 3: 266-272; Karlin and Altschul, 1993, Applications and statistics for multiple high-scoring segments in molecular sequences, Proc. Natl. Acad. Sci. USA 90: 5873-5877; all of which are incorporated by reference herein).

In all search programs in the suite the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Analogues are defined as proteins with similar three-dimensional structures and/or functions with little evidence of a common ancestor at a sequence level.

Homologues are proteins with evidence of a common ancestor, i.e. likely to be the result of evolutionary divergence and are divided into remote, medium and close sub-divisions based on the degree (usually expressed as a percentage) of sequence identity.

A homologue is defined herein as a protein with at least about 20%, e.g. at least 25%, at least 30%, at least 35%, at least 40%, at least 50%, preferably at least 60% sequence, more preferably at least 70%, more preferably at least 80%, more preferably at least 90% and more preferably at least 95% identity to human solAC. This includes polymorphic forms of solAC and species forms including those species referred to in Tables 12 and 13 below.

Tables 12 and 13 shows percentage identity between mammalian soluble adenylate cyclases.

TABLE 12
Percentage identity between mammalian soluble adenylate cyclases.
Comparison has been done along entire sequence:

| | Homo | Oryctolagus | Rattus | Mus | Pan | Canis |
|---|---|---|---|---|---|---|
| | sapiens | cuniculus | norvegicus | musculus | troglodytes | familiaris |
| Homo | 100.0 | 85.4 | 83.2 | 72.6 | 57.7 | 17.0 |
| sapiens | | | | | | |
| Oryctolagus | 85.4 | 100.0 | 91.1 | 74.5 | 50.9 | 17.9 |
| cuniculus | | | | | | |
| Rattus | 83.2 | 91.1 | 100.0 | 79.9 | 49.4 | 18.1 |
| norvegicus | | | | | | |
| Mus | 72.6 | 74.5 | 79.7 | 100.0 | 47.0 | 17.9 |
| musculus | | | | | | |
| Pan | 57.7 | 50.9 | 49.4 | 47.0 | 100.0 | 16.0 |
| troglodytes | | | | | | |
| Canis | 17.0 | 17.9 | 18.1 | 17.9 | 16.0 | 100.0 |
| familiaris | | | | | | |

TABLE 13
Percentage identity between mammalian soluble adenylate cyclases.
Comparison has been done between sequence of catalytic domains alone.

| | Homo | Oryctolagus | Rattus | Mus | Pan | Canis |
|---|---|---|---|---|---|---|
| | sapiens | cuniculus | norvegicus | musculus | troglodytes | familiaris |
| Homo | 100.0 | 88.8 | 86.7 | 86.2 | 69.5 | 34.3 |
| sapiens | | | | | | |
| Oryctolagus | 88.8 | 100.0 | 92.7 | 92.0 | 63.1 | 34.3 |
| cuniculus | | | | | | |
| Rattus | 86.7 | 92.7 | 100.0 | 98.7 | 62.9 | 34.8 |
| norvegicus | | | | | | |
| Mus | 86.2 | 92.0 | 98.7 | 100.0 | 62.5 | 35.0 |
| musculus | | | | | | |
| Pan | 69.5 | 63.1 | 62.9 | 62.5 | 100.0 | 28.9 |
| troglodytes | | | | | | |
| Canis | 34.3 | 34.3 | 34.8 | 35.0 | 28.9 | 100.0 |
| familiaris | | | | | | |

There are two types of homologue: orthologues and paralogues. Orthologues are defined as homologous genes in different organisms, i.e. the genes share a common ancestor coincident with the speciation event that generated them. Paralogues are defined as homologous genes in the same organism derived from a gene/chromosome/genome duplication, i.e. the common ancestor of the genes occurred since the last speciation event.

The homologues could also be polymorphic forms of solAC such as alleles or mutants as described in section (A) or chimeras as described in section (D).

Once the amino acid sequences of the polypeptides with known and unknown structures are aligned, the structures of the conserved amino acids in a computer representation of the polypeptide with known structure are transferred to the corresponding amino acids of the polypeptide whose structure is unknown. For example, a tyrosine in the amino acid sequence of known structure may be replaced by a phenylalanine, the corresponding homologous amino acid in the amino acid sequence of unknown structure.

The structures of amino acids located in non-conserved regions may be assigned manually by using standard peptide geometries or by molecular simulation techniques, such as molecular dynamics. The final step in the process is accomplished by refining the entire structure using molecular dynamics and/or energy minimization.

Homology modelling as such is a technique that is well known to those skilled in the art (see e.g. Greer, (Science, Vol. 228, (1985), 1055), and Blundell et al., (Eur. J. Biochem, Vol. 172, (1988), 513). The techniques described in these references, as well as other homology modelling techniques, generally available in the art, may be used in performing the present invention.

Thus the invention provides a method of homology modelling comprising the steps of:
(a) aligning a representation of an amino acid sequence of a target solAC protein of unknown three-dimensional structure with the amino acid sequence of the solAC of Table 1, Table 2, Table 3, Table 4 or Table 5, optionally varied by a root mean square deviation of not more than 1.5 Å, or selected coordinates thereof, to match homologous regions of the amino acid sequences;
(b) modelling the structure of the matched homologous regions of said target solAC of unknown structure on the corresponding regions of the solAC structure as defined by Table 1, Table 2, Table 3, Table 4 or Table 5, optionally varied by a root mean square deviation of not more than 1.5 Å, or selected coordinates thereof; and
(c) determining a conformation for said target solAC of unknown structure which substantially preserves the structure of said matched homologous regions.

A conformation for said target solAC of unknown structure will for example be one in which favourable interactions are formed within the target adenylate cyclase of unknown structure and/or so that a low energy conformation is formed.

Preferably one or all of steps (a) to (c) are performed by computer modelling.

The coordinate data of Table 1, Table 2, Table 3, Table 4 or Table 5, or selected coordinates thereof, will be particularly advantageous for homology modelling of other adenylate cyclases.

The aspects of the invention described herein which utilise the solAC structure in silico may be equally applied to homologue models of adenylate cyclase obtained by the above aspect of the invention, and this application forms a further aspect of the present invention. Thus having determined a conformation of an adenylate cyclase by the method described above, such a conformation may be used in a computer-based method of rational drug design as described herein.

### E. Structure Solution

The atomic coordinate data of solAC can also be used to solve the crystal structure of other target adenylate cyclase proteins including other crystal forms of solAC, mutants, co-complexes of solAC, where X-ray diffraction data or NMR spectroscopic data of these target adenylate cyclase proteins has been generated and requires interpretation in order to provide a structure.

In the case of solAC, this protein may crystallize in more than one crystal form. The data of Table 1, Table 2, Table 3, Table 4 or Table 5, or selected coordinates thereof, as provided by this invention, are particularly useful to solve the structure of those other crystal forms of solAC. It may also be used to solve the structure of solAC mutants, solAC co-complexes, or of the crystalline form of any other protein with significant amino acid sequence homology to any functional domain of solAC.

In the case of other target adenylate cyclase proteins, particularly the homologous mammalian soluble adenylate cyclases such as those from the species referred to in Tables 12 and 13, the present invention allows the structures of such targets to be obtained more readily where raw X-ray diffraction data is generated.

Thus, where X-ray crystallographic or NMR spectroscopic data is provided for a target protein adenylate cyclase or solAC of unknown three-dimensional structure, the atomic coordinate data derived from Table 1, Table 2, Table 3, Table 4 or Table 5, may be used to interpret that data to provide a likely structure for the other adenylate cyclase by techniques which are well known in the art, e.g. phasing in the case of X-ray crystallography and assisting peak assignments in NMR spectra.

One method that may be employed for these purposes is molecular replacement. In this method, the unknown crystal structure, whether it is another crystal form of solAC, a solAC mutant, a solAC chimera or a solAC co-complex, or the crystal of a target adenylate cyclase protein with amino acid sequence homology to any functional domain of solAC, may be determined using the solAC structure coordinates of all or part of Table 1, Table 2, Table 3, Table 4 or Table 5 of this invention. This method will provide an accurate structural form for the unknown crystal more quickly and efficiently than attempting to determine such information ab initio.

Examples of computer programs known in the art for performing molecular replacement are CNX (Brunger A. T.; Adams P. D.; Rice L. M., Current Opinion in Structural Biology, Volume 8, Issue 5, October 1998, Pages 606-611 (also commercially available from Accelrys San Diego, Calif.), MOLREP (A. Vagin, A. Teplyakov, MOLREP: an automated program for molecular replacement, J. Appl. Cryst. (1997) 30, 1022-1025, part of the CCP4 suite) or AMoRe (Navaza, J. (1994). AMoRe: an automated package for molecular replacement. Acta Cryst. A50, 157-163).

Thus, in a further aspect of the invention provides a method for determining the structure of a protein, which method comprises;
providing the co-ordinates of Table 1, Table 2, Table 3, Table 4 or Table 5, optionally varied by an rmsd of less than 1.5 Å, or selected coordinates thereof; and
either (a) positioning said co-ordinates in the crystal unit cell of said protein so as to provide a structure for said protein, or (b) assigning NMR spectra peaks of said protein by manipulating said co-ordinates.

Preferably the coordinates of Table 1, Table 2, Table 3, Table 4 or Table 5 or selected coordinates thereof, include coordinates of atoms of the amino acid residues set out in any one of Tables 6 to 11, such as Tables 6, 7 or 11. Preferred selected coordinates of each Table, or combinations of coordinates from two or more Tables are as described elsewhere herein.

The invention may also be used to assign peaks of NMR spectra of such proteins, by manipulation of the data of Table 1, Table 2, Table 3, Table 4 or Table 5.

In a preferred aspect of this invention the co-ordinates are used to solve the structure of target adenylate cyclase, particularly homologues of solAC for example other adenylate cyclases, and in particular isoforms of solAC from different species.

### F. Computer Systems.

In another aspect, the present invention provides systems, particularly a computer system, intended to generate structures and/or perform optimisation of ligands which interact with solAC, solAC homologues or analogues, complexes of solAC with ligands, or complexes of solAC homologues or analogues with ligands, the system containing computer-readable data comprising one or more of:
(a) solAC co-ordinate data of Table 1, Table 2, Table 3, Table 4 or Table 5 said data defining the three-dimensional structure of solAC catalytic domain, or selected coordinates thereof;
(b) atomic coordinate data of a target adenylate cyclase protein generated by homology modelling of the target based on the coordinate data of Table 1, Table 2, Table 3, Table 4 or Table 5;
(c) atomic coordinate data of a target adenylate cyclase protein generated by interpreting X-ray crystallographic data or NMR data by reference to the co-ordinate data of Table 1, Table 2, Table 3, Table 4 or Table 5;
(d) structure factor data derivable from the atomic coordinate data of (b) or (c).
   and
(e) atomic coordinate data of Table 1, Table 2, Table 3, Table 4 or Table 5, optionally varied by an rmsd of less than 1.5 Å, or selected coordinates thereof.

For example the computer system may comprise: (i) a computer-readable data storage medium comprising data storage material encoded with the computer-readable data; (ii) a working memory for storing instructions for processing said computer-readable data; and (iii) a central-processing unit coupled to said working memory and to said computer-readable data storage medium for processing said computer-readable data and thereby generating structures and/or performing rational drug design. The computer system may further comprise a display coupled to said central-processing unit for displaying said structures.

The invention also provides such systems containing atomic coordinate data of target adenylate cyclase proteins wherein such data has been generated according to the methods of the invention described herein based on the starting data provided the data of Table 1, Table 2, Table 3, Table 4 or Table 5 or selected coordinates thereof.

Such data is useful for a number of purposes, including the generation of structures to analyse the mechanisms of action of adenylate cyclase proteins and/or to perform rational drug design of ligands which interact with solAC, such as compounds which are metabolised by adenylate cyclases.

In another aspect, the invention provides computer-readable storage medium, comprising a data storage material encoded with computer readable data, wherein the data are defined by the structure coordinates of the solAC protein of Table 1, Table 2, Table 3, Table 4 or Table 5 or selected coordinates thereof, or a homologue of solAC, wherein said homologue comprises backbone atoms that have a root mean square deviation from the backbone atoms of said Table 1, Table 2, Table 3, Table 4 or Table 5 or selected coordinates thereof of less than 1.5 Å.

In a further aspect, there is provided a computer-readable storage medium comprising a data storage material encoded with a first set of computer-readable data comprising a Fourier transform of at least a portion of the structural coordinates for the solAC protein defined by the structure of Table 1, Table 2, Table 3, Table 4 or Table 5, optionally varied by an rmsd of less than 1.5 Å, or selected coordinates thereof;
which data, when combined with a second set of machine readable data comprising an X-ray diffraction pattern of a molecule or molecular complex of unknown structure, using a machine programmed with the instructions for using said first set of data and said second set of data, can determine at least a portion of the structure coordinates corresponding to the second set of machine readable data.

As used herein, “computer readable media” refers to any medium or media, which can be read and accessed directly by a computer. Such media include, but are not limited to: magnetic storage media such as floppy discs, hard disc storage medium and magnetic tape; optical storage media such as optical discs or CD-ROM; electrical storage media such as RAM and ROM; and hybrids of these categories such as magnetic/optical storage media.

By providing such computer readable media, the atomic coordinate data of the invention can be routinely accessed to model adenylate cyclases or selected coordinates thereof. For example, RASMOL (Sayle et al., TIBS, Vol. 20, (1995), 374) is a publicly available computer software package, which allows access and analysis of atomic coordinate data for structure determination and/or rational drug design.

As used herein, “a computer system” refers to the hardware means, software means and data storage means used to analyse the atomic coordinate data of the invention. The minimum hardware means of the computer-based systems of the present invention comprises a central processing unit (CPU), input means, output means and data storage means. Desirably a monitor is provided to visualize structure data. The data storage means may be RAM or means for accessing computer readable media of the invention. Examples of such systems are microcomputer workstations available from Silicon Graphics Incorporated and Sun Microsystems running Unix based, Windows XP or IBM OS/2 operating systems.

The invention also provides a computer-readable data storage medium comprising a data storage material encoded with a first set of computer-readable data comprising the solAC coordinates of Table 1, Table 2, Table 3, Table 4 or Table 5 or selected coordinates thereof; which, when combined with a second set of machine readable data comprising an X-ray diffraction pattern of a molecule or molecular complex of unknown structure, using a machine programmed with the instructions for using said first set of data and said second set of data, can determine at least a portion of the electron density corresponding to the second set of machine readable data.

A further aspect of the invention provides a method of providing data for generating structures and/or performing optimisation of ligands which interact with solAC, solAC homologues or analogues, complexes of solAC with ligands, or complexes of solAC homologues or analogues with ligands, the method comprising:
(i) establishing communication with a remote device containing
   (a) computer-readable data comprising atomic coordinate data of Table 1, Table 2, Table 3, Table 4 or Table 5, optionally varied by an rmsd of less than 1.5 Å, or selected coordinates thereof, said data defining the three-dimensional structure of solAC catalytic domain, or selected coordinates thereof;
   (b) atomic coordinate data of a target adenylate cyclase homologue or analogue generated by homology modelling of the target based on the data (a);
   (c) atomic coordinate data of a protein generated by interpreting X-ray crystallographic data or NMR data by reference to the data of Table 1, Table 2, Table 3, Table 4 or Table 5 and
   (d) structure factor data derivable from the atomic coordinate data of (a) or (c); and
(ii) receiving said computer-readable data from said remote device.

In a further aspect, the invention provides the use of a computer for producing a three-dimensional representation of a solAC structure or a solAC-ligand complex wherein the solAC structure is of Table 1, Table 2, Table 3, Table 4 or Table 5, optionally varied by a root mean square deviation of not more than 1.5 Å, or selected coordinates thereof, wherein said computer comprises:
(i) a machine-readable data storage medium comprising a data storage material encoded with machine-readable data, wherein said data comprise the structure of Table 1, Table 2, Table 3, Table 4 or Table 5, optionally varied by a root mean square deviation of not more than 1.5 Å, or selected coordinates thereof;
(ii) instructions for processing said machine-readable data into said three-dimensional representation.

The computer may further comprise a display for displaying said three-dimensional representation.

The computer-readable data received from said remote device, particularly when in the form of the atomic coordinate data of Table 1, Table 2, Table 3, Table 4 or Table 5, optionally varied by an rmsd of less than 1.5 Å, or selected coordinates thereof, may be used in the methods of the invention described herein, e.g. for the analysis of a ligand structure with a solAC structure.

Thus the remote device may comprise e.g. a computer system or computer readable media of one of the previous aspects of the invention. The device may be in a different country or jurisdiction from where the computer-readable data is received.

The communication may be via the internet, intranet, e-mail etc, transmitted through wires or by wireless means such as by terrestrial radio or by satellite. Typically the communication will be electronic in nature, but some or all of the communication pathway may be optical, for example, over optical fibers.

### G. Uses of the Structures of the Invention.

The crystal structures obtained according to the present invention as well as the structures of target adenylate cyclase proteins obtained in accordance with the methods described herein, may be used in several ways for drug design. For example, the design of ligands selective for solAC, such as the nucleotide binding region of solAC, may be undertaken by reference to the coordinate data of Table 1, Table 2, Table 3, Table 4 or Table 5 or selected coordinates thereof. In one aspect, the non-cyclizable nucleotide analogue AMPCPP is able to bind in the nucleotide binding pocket of many different adenylate cyclases. The use of the present structure of the solAC catalytic domain permits the design of structures which will have greater specificity for solAC compared to other adenylate cyclases.

More generally, the structures of the invention may be used in the provision, design, modification or analysis of modulators of solAC. As used herein, reference to a “modulator” of solAC is intended to refer to a ligand which causes a change (i.e a modulation) in the level of biological activity of the solAC protein. Thus modulation encompasses physiological changes which effect an increase or decrease in solAC activity. In the former case, the modulation may be described as an activation; in the latter, an inhibition. The modulation may arise directly as a result of ligand binding to the ATP binding site of solAC or indirectly, e.g. by ligand binding to any site of the solAC such that the activity of solAC or its interactions with other proteins are affected. Such interactions may include interaction with other gene products or proteins which localise the solAC enzyme to specific organelles (for example mitochondria, centrioles, mitotic spindles, nuclei) or brings about the interaction between solAC and effector molecules (for example PKA) or at the level of enzyme activity (for example by allosteric mechanisms, competitive inhibition, active-site inactivation, perturbation of feedback inhibitory pathways etc). Thus, modulation may imply over- or under-expression of the solAC brought about by such mechanisms, as well as hyper-(or hypo-)activity due to ligand binding at the ATP binding site, or at the bicarbonate binding site, or at any of the other sites identified herein. The terms “modulator”, “modulation” and “modulate” as used in relation to solAC are to be interpreted accordingly.

For example, information on the binding orientation of a ligand in the binding pocket can be determined by either co-crystallization, soaking or computationally docking the ligand. This will guide specific modifications to the chemical structure designed to mediate or control the interaction of the ligand with the protein. Such modifications can be designed with an aim to modify the interaction of the ligand with solAC and so improve its therapeutic action.

Thus, the determination of the three-dimensional structure of solAC provides a basis for the design of new ligands, e.g. chemical compounds, which interact with solAC. For example, knowing the three-dimensional structure of solAC, computer modelling programs may be used to design different molecules expected to interact with possible or confirmed active sites, such as binding sites or other structural or functional features of solAC.

### (i) Obtaining and Analysing Crystal Complexes.

In one approach, the structure of a ligand bound to a solAC may be determined by experiment. This will provide a starting point in the analysis of the ligand bound to solAC, thus providing those of skill in the art with a detailed insight as to how that particular ligand interacts with solAC and the mechanism by which, for example, it competes with ATP for the binding pocket.

Many of the techniques and approaches applied to structure-based drug design described above rely at some stage on X-ray analysis to identify the binding position of a ligand in a ligand-protein complex. A common way of doing this is to perform X-ray crystallography on the complex, produce a difference Fourier electron density map, and associate a particular pattern of electron density with the ligand. However, in order to produce the map (as explained e.g. by Blundell et al., in Protein Crystallography, Academic Press, New York, London and San Francisco, (1976)), it is necessary to know beforehand the protein 3D structure (or at least a set of structure factors for the protein crystal). Therefore, determination of the solAC structure also allows difference Fourier electron density maps of solAC-ligand complexes to be produced, determination of the binding position of the drug and hence may greatly assist the process of rational drug design.

Accordingly, the invention provides a method for determining the structure of a ligand bound to solAC, said method comprising:
providing a crystal of the solAC protein;
soaking the crystal with the ligand to form a complex; and
determining the structure of the complex by employing the data of Table 1, Table 2, Table 3, Table 4 or Table 5, optionally varied by a root mean square deviation of not more than 1.5 Å, or selected coordinates thereof.

Alternatively, the solAC and catalytic domain and ligand may be co-crystallized. Purified protein samples are incubated over a period of time (usually >1 hr) with a potential ligand. The protein ligand complex can then be screened for crystallization conditions. Alternatively, protein crystals containing one ligand can be back-soaked to remove this ligand by placing the crystals into a stabilising solution in which the ligand is not present. The resultant crystals can then be transferred into a second solution containing a different ligand.

Thus the invention provides a method for determining the structure of a ligand bound to solAC, said method comprising;
mixing solAC protein with the ligand;
crystallizing a solAC protein-ligand complex; and
determining the structure of the complex by employing the data of Table 1, Table 2, Table 3, Table 4 or Table 5, optionally varied by an rmsd of less than 1.5 Å, or selected coordinates thereof.

A mixture of compounds may be soaked or co-crystallized with the crystal, wherein only one or some of the compounds may be expected to bind to the solAC. The mixture of compounds may comprise a ligand known to bind to solAC. As well as the structure of the complex, the identity of the complexing compound(s) is/are then determined.

The method may comprise the further steps of: (a) obtaining or synthesising said candidate ligand; (b) forming a complex of solAC and said candidate ligand; and (c) analysing said complex by X-ray crystallography or NMR spectroscopy to determine the ability of said candidate ligand to interact with solAC.

The analysis of such structures may employ (i) X-ray crystallographic diffraction data from the complex and (ii) a three-dimensional structure of solAC, or at least selected coordinates thereof, to generate a difference Fourier electron density map of the complex, the three-dimensional structure being defined by atomic coordinate data of Table 1, Table 2, Table 3, Table 4 or Table 5 or selected coordinates thereof. The difference Fourier electron density map may then be analysed.

Therefore, such complexes can be crystallized and analysed using X-ray diffraction methods, e.g. according to the approach described by Greer et al., (J. of Medicinal Chemistry, Vol. 37, (1994), 1035-1054), and difference Fourier electron density maps can be calculated based on X-ray diffraction patterns of soaked or co-crystallized solAC and the solved structure of uncomplexed solAC. These maps can then be analysed e.g. to determine whether and where a particular ligand binds to solAC and/or changes the conformation of solAC.

Electron density maps can be calculated using programs such as those from the CCP4 computing package (Collaborative Computational Project 4. The CCP4 Suite: Programs for Protein Crystallography, Acta Crystallographica, D50, (1994), 760-763.). For map visualization and model building programs such as “O” (Jones et al., Acta Crystallographica, A47, (1991), 110-119) can be used.

In addition, in accordance with this invention, solAC mutants may be crystallized in co-complex with known solAC ligands or novel ligands. The crystal structures of a series of such complexes may then be solved by molecular replacement and compared with that of the solAC structure of Table 1, Table 2, Table 3, Table 4 or Table 5 or selected coordinates thereof. Potential sites for modification within the various binding sites of the enzyme may thus be identified. This information provides an additional tool for determining the most efficient binding interactions, for example, increased hydrophobic interactions, between solAC and a ligand.

All of the complexes referred to above may be studied using well-known X-ray diffraction techniques and may be refined against 1.5 to 3.5 Å resolution X-ray data to an R value of about 0.30 or less using computer software, such as CNX (Brunger et al., Current Opinion in Structural Biology, Vol. 8, Issue 5, October 1998, 606-611, and commercially available from Accelrys, San Diego, Calif.), and as described by Blundell et al, (1976) and Methods in Enzymology, vol. 114 & 115, H. W. Wyckoff et al., eds., Academic Press (1985).

This information may thus be used to optimise known classes of solAC ligands, and more importantly, to design and synthesize novel classes of solAC ligands, particularly inhibitors or activators, and to design drugs with modified solAC interactions.

### (ii) In Silico Analysis and Design

Although the invention will facilitate the determination of actual crystal structures comprising a solAC catalytic domain and a ligand, which interacts with the solAC catalytic domain, current computational techniques provide a powerful alternative to the need to generate such crystals and generate and analyse diffraction date. Accordingly, a particularly preferred aspect of the invention relates to computer based (“in silico”) methods directed to the analysis and development of ligands which interact with solAC structures of the present invention.

Determination of the three-dimensional structure of the solAC catalytic domain provides important information about the binding sites of solAC, particularly when comparisons are made with similar enzymes. This information may then be used for rational design and modification of solAC ligands, e.g. by computational techniques which identify possible binding ligands for the binding sites, by enabling linked-fragment approaches to drug design, and by enabling the identification and location of bound ligands using X-ray crystallographic analysis. These techniques are discussed in more detail below.

Thus as a result of the determination of the solAC three-dimensional structure, more purely computational techniques for rational drug design may also be used to design structures whose interaction with solAC is better understood (for an overview of these techniques see e.g. Walters et al (Drug Discovery Today, Vol. 3, No. 4, (1998), 160-178; Abagyan, R.; Totrov, M. Curr. Opin. Chem. Biol. 2001, 5, 375-382). For example, automated ligand-receptor docking programs (discussed e.g. by Jones et al. in Current Opinion in Biotechnology, Vol. 6, (1995), 652-656 and Halperin, I.; Ma, B.; Wolfson, H.; Nussinov, R. Proteins 2002, 47, 409-443), which require accurate information on the atomic coordinates of target receptors may be used.

The aspects of the invention described herein which utilize the solAC structure in silico may be equally applied to both the solAC structure of Table 1, Table 2, Table 3, Table 4 or Table 5 or selected coordinates thereof and the models of target adenylate cyclase proteins obtained by other aspects of the invention. Thus having determined a conformation of a solAC by the method described above, such a conformation may be used in a computer-based method of rational drug design as described herein. In addition the availability of the structure of the solAC will allow the generation of highly predictive pharmacophore models for virtual library screening or ligand design.

Accordingly, the invention provides a method for the analysis of the interaction of a ligand with a solAC structure, which comprises:
providing a solAC structure which is of Table 1, Table 2, Table 3, Table 4 or Table 5, optionally varied by a root mean square deviation of not more than 1.5 Å, or selected coordinates thereof;
providing a ligand to be fitted to said solAC structure or selected coordinates thereof; and
fitting the ligand to said solAC structure.

This method of the invention is generally applicable for the analysis of known ligands of solAC, the development or discovery of ligands of solAC, the modification of ligands of solAC e.g. to improve or modify one or more of their properties, and the like.

In performing the methods of the invention, the solAC structure of Table 1, Table 2, Table 3, Table 4 or Table 5, or selected coordinates thereof, may be represented in a computer model in accordance with standard techniques known as such in the art. Those of skill in the art will be familiar with means of providing three-dimensional representations of proteins in such methods. Suitable models include (a) a wire-frame model; (b) a chicken-wire model; (c) a ball-and-stick model; (d) a space-filling model; (e) a stick-model; (f) a ribbon model; (g) a snake model; (h) an arrow and cylinder model; (i) an electron density map; or (j) a molecular surface model.

The selected coordinates may be from one or more side chain or main chain atoms from some or all of the amino acids listed in any one of Tables 6 to 11, such as Tables 6, 7 or 11, with preferred numbers and combinations of such coordinates being as described elsewhere herein.

In an alternative aspect, the method of the invention may utilize the coordinates of atoms of interest of the solAC ligand binding region, which are in the vicinity of a putative ligand structure, for example within 10-25 Å of the catalytic regions or within 5-10 Å of a ligand bound, in order to model the pocket in which the structure binds. These coordinates may be used to define a space, which is then analysed in silico as described above.

In practice, it will be desirable to model a sufficient number of atoms of the solAC as defined by the coordinates of Table 1, Table 2, Table 3, Table 4 or Table 5 or selected coordinates thereof, which represent a binding pocket, e.g. the atoms of the residues identified in any one of Tables 6 to 11, such as Tables 6, 7 or 11. Preferred selected coordinates of each Table, or combinations of coordinates from two or more Tables are as described elsewhere herein. Binding pockets and other features of the interaction of solAC with ligand are described in the accompanying example.

Although every different ligand bound by solAC may interact with different parts of the binding pocket of the protein, the structure of this solAC allows the identification of a number of particular sites which are likely to be involved in many of the interactions of solAC with a drug candidate. The residues are set out in Tables 6 to 11, such as Tables 6, 7, or 11. Thus in this aspect of the invention, the selected coordinates may comprise coordinates of some or all of these residues. Preferred selected coordinates of each Table, or combinations of coordinates from two or more Tables are as described elsewhere herein.

In order to provide a three-dimensional structure of ligands to be fitted to a solAC structure of the invention, the ligand structure may be modelled in three dimensions using commercially available software for this purpose or, if its crystal structure is available, the coordinates of the structure may be used to provide a representation of the ligand for fitting to a solAC structure of the invention.

Newly designed ligand structures may be synthesised and their interaction with solAC may be determined or predicted as to how the newly designed structure is bound by said solAC structure. This process may be iterated so as to further alter the interaction between it and the solAC.

By “fitting”, is meant determining by automatic, or semi-automatic means, interactions between one or more atoms of a candidate molecule and at least one atom of a solAC structure of the invention, and calculating the extent to which such interactions are stable. Interactions include attraction and repulsion, brought about by charge, steric, lipophilic, considerations and the like. Charge and steric interactions of this type can be modeled computationally. An example of such computation would be via a force field such as Amber (Cornell et al. A Second Generation Force Field for the Simulation of Proteins, Nucleic Acids, and Organic Molecules, Journal of the American Chemical Society, (1995), 117(19), 5179-97) which would assign partial charges to atoms on the protein and ligand and evaluate the electrostatic interaction energy between a protein and ligand atom using the Coulomb potential. The Amber force field would also assign van der Waals energy terms to assess the attractive and repulsive steric interactions between two atoms. Lipophilic interactions can be modeled using a variety of means. For example the ChemScore function (Eldridge M D; Murray C W; Auton T R; Paolini G V; Mee R P Empirical scoring functions: I. The development of a fast empirical scoring function to estimate the binding affinity of ligands in receptor complexes, Journal of computer-aided molecular design (1997 September), 11 (5), 425-45) assigns protein and ligand atoms as hydrophobic or polar, and a favorable energy term is specified for the interaction between two hydrophobic atoms. Other methods of assessing the hydrophobic contributions to ligand binding are available and these would be known to one skilled in the art. Other methods of assessing interactions are available and would be known to one skilled in the art of designing molecules. Various computer-based methods for fitting are described further herein.

More specifically, the interaction of a ligand with a solAC structure of the invention can be examined through the use of computer modelling using a docking program such as GOLD (Jones et al., J. Mol. Biol., 245, 43-53 (1995), Jones et al., J. Mol. Biol., 267, 727-748 (1997)), GRAMM (Vakser, I. A., Proteins, Suppl., 1:226-230 (1997)), DOCK (Kuntz et al, (1982) J. Mol. Biol., 161, 269-288; Makino et al, (1997) J Comput. Chem., 18, 1812-1825), AUTODOCK (Goodsell et al, (1990) Proteins, 8, 195-202, Morris et al, (1998) J. Comput. Chem., 19, 1639-1662.), FlexX, (Rarey et al, (1996) J. Mol. Biol., 261, 470-489) or ICM (Abagyan et al, (1994) J. Comput. Chem., 15, 488-506). This procedure can include computer fitting of ligands to solAC to ascertain how well the shape and the chemical structure of the ligand will bind to the solAC.

Also computer-assisted, manual examination of the active site structure of solAC may be performed. The use of programs such as GRID (Goodford, (1985) J. Med. Chem., 28, 849-857)—a program that determines probable interaction sites between molecules with various functional groups and an enzyme surface—may also be used to analyse the active site to predict, for example, the types of modifications which will alter the rate of metabolism of a ligand.

Computer programs can be employed to estimate the attraction, repulsion, and steric hindrance of the two binding partners (i.e. a solAC structure and a ligand).

If more than one solAC active site pocket is characterized and a plurality of respective smaller ligands are designed or selected, a ligand may be formed by linking the respective small ligands into a larger ligand, which maintains the relative positions and orientations of the respective ligands at the active sites. The larger ligand may be formed as a real molecule or by computer modelling.

Detailed structural information can then be obtained about the binding of the ligand to solAC, and in the light of this information adjustments can be made to the structure or functionality of the ligand, e.g. to alter its interaction with solAC. The above steps may be repeated and re-repeated as necessary.

In another aspect, the present invention provides a method for identifying a ligand for modulating the activity of solAC, comprising the steps of: (a) employing three-dimensional atomic coordinate data according to Table 1, Table 2, Table 3, Table 4 or Table 5, optionally varied by a root mean square deviation of not more than 1.5 Å, or selected coordinates thereof, to characterise at least one solAC binding site and preferably a plurality of solAC binding sites; (b) providing the structure of a candidate ligand; (c) fitting the candidate ligand to the binding site or sites; and (d) selecting a candidate ligand which fits the site or sites.

In one embodiment a plurality of candidate ligands are screened or interrogated for interaction with the binding sites. In one example, step (b) involves providing the structures of the candidate ligands, each of which is then fitted in step (c) to computationally screen a database of compounds (such as the Cambridge Structural Database) for interaction with the binding sites, i.e. the candidate ligand may be selected by computationally screening a database of ligands for interaction with the binding sites (see Martin, J. Med. Chem., vol 35, 2145-2154 (1992)). In another example, a 3-D descriptor for the ligand is derived, the descriptor including e.g. geometric and functional constraints derived from the architecture and chemical nature of the binding cavity or cavities. The descriptor may then be used to interrogate the compound database, the identified ligand being a compound which matches with the features of the descriptor. In effect, the descriptor is a type of virtual pharmacophore.

As indicated above, ligands which may be fitted to the solAC structure of the invention, include compounds under development as potential pharmaceutical agents. The agents may be fitted in order to determine how the action of solAC modifies the agent and to provide a basis for modelling candidate ligands, which for example bind to solAC in competition with ATP.

Having obtained and characterized a ligand compound according to the invention, the invention further provides a method for modulating the activity of solAC which method comprises: (a) providing solAC under conditions where, in the absence of ligand, the solAC is able to bind ATP; (b) providing a ligand compound; and (c) determining the extent to which the ability of solAC to bind ATP is altered by the presence of said compound, e.g. by competition.

(iii) Analysis and Modification of Ligands

Where a ligand is known to modulate or suspected of modulating the action of solAC, the structure of the ligand may be modelled in order to better determine residues of solAC which interact with the ligand. The present invention also provides a process for predicting further ligands which act as solAC ligands, which method comprises:
fitting said ligand to the three-dimensional structure of the solAC catalytic domain, or selected co-ordinates thereof, the three-dimensional structure being defined by the co-ordinates of Table 1, Table 2, Table 3, Table 4 or Table 5, optionally varied by an rmsd of less than 1.5 Å, or selected coordinates thereof;
determining or predicting how said ligand binds to said catalytic domain; and
modifying the ligand structure so as to increase or decrease its interaction with the catalytic domain.

Typically the ligand will be modified with the aim of producing a more effective ligand than the starting ligand, or of producing a more pharmaceutically acceptable ligand.

It would be understood by those of skill in the art that modification of the structure will usually occur in silico, allowing predictions to be made as to how the modified structure interacts with the solAC.

Greer et al. (Greer et al. (1994), J. of Medicinal Chemistry, 37, 1035-1054) describes an iterative approach to ligand design based on repeated sequences of computer modelling, protein-ligand complex formation and X-ray crystallographic or NMR spectroscopic analysis. Thus novel thymidylate synthase inhibitor series were designed de novo by Greer et al., and solAC ligands may also be designed or modified in the this way. More specifically, using e.g. GRID on the solved structure of solAC, a ligand for solAC may be designed that complements the functionalities of the solAC binding sites. Alternatively a ligand for solAC may be modified such that it complements the functionalities of the solAC binding sites better or less well. The ligand can then be synthesised, formed into a complex with solAC, and the complex then analysed by X-ray crystallography to identify the actual position of the bound ligand. The structure and/or functional groups of the ligand can then be adjusted, if necessary, in view of the results of the X-ray analysis, and the synthesis and analysis sequence repeated until an optimised ligand is obtained. Related approaches to structure-based drug design are also discussed in Bohacek et al. (1996) Medicinal Research Reviews, 16, 3-50. Design of a ligand with alternative solAC properties using structure based drug design may also take into account the requirements for high affinity to a second, target protein. Gschwend et al., (Gschwend et al. (1999), Bioorganic & Medicinal Chemistry Letters, 9, 307-312) and Bayley et al., (Bayley et al. (1997) Proteins: Structure, Function and Genetics, 29, 29-67) describe approaches where structure based drug design is used to reduce affinity to one protein whilst maintaining affinity for a target protein.

The modifications will be those conventional in the art known to the skilled medicinal chemist, and will include, for example, substitutions or removal of groups containing residues which interact with the amino acid side chain groups of a solAC structure of the invention. For example, the replacements may include the addition or removal of groups in order to decrease or increase the charge of a group in a test compound, the replacement of a group to increase or decrease the size of the group in a test compound, the replacement of a charge group with a group of the opposite charge, or the replacement of a hydrophobic group with a hydrophilic group or vice versa. It will be understood that these are only examples of the type of substitutions considered by medicinal chemists in the development of new pharmaceutical compounds and other modifications may be made, depending upon the nature of the starting compound and its activity.

Where a potential modified ligand has been developed by fitting a starting ligand to the solAC structure of the invention and predicting from this a modified ligand, the invention further includes the step of synthesizing the modified ligand and testing it in a in vivo or in vitro biological system in order to determine its activity and/or its effectiveness as a ligand for solAC.

The above-described processes of the invention may be iterated in that the modified ligand may itself be the basis for further ligand design. The above-described processes may also be used to modify a ligand which interacts with a second ligand within the solAC binding pocket.

### (iv) Analysis of Ligands in Binding Pocket Regions

In one embodiment, the present invention provides a method for modifying the structure of a ligand, which method comprises:
fitting said ligand to the three-dimensional structure of the solAC catalytic domain, or selected co-ordinates thereof, the three-dimensional structure being defined by the co-ordinates of Table 1, Table 2, Table 3, Table 4 or Table 5, optionally varied by an rmsd of less than 1.5 Å, or selected coordinates thereof; and
modifying the ligand structure so as to increase or decrease its interaction with the catalytic domain.
wherein said fitting is to the ligand-binding region defined as including at least one coordinate of an amino acid residue set out in any one of Tables 6 to 11, such as Tables 6, 7, or 11. Preferred selected coordinates of each Table, or combinations of coordinates from two or more Tables are as described elsewhere herein. Typically the ligand will be modified with the aim of improving its inhibitory effectiveness, or its pharmaceutical acceptability.

For the avoidance of doubt, the term “modifying” is used as defined in the preceding subsection, and once such a ligand has been developed it may be synthesised and tested also as described above.

### (vi) Fragment Linking and Growing.

The provision of the crystal structures of the invention will also allow the development of ligands which interact with the binding pocket regions the solAC catalytic domain (for example to act as ligands, particularly inhibitors or activators of a solAC) based on a fragment linking or fragment growing approach.

For example, the binding of one or more ligand fragments can be determined in the protein binding pocket by X-ray crystallography. Ligand fragments are typically compounds with a molecular weight between 100 and 200 Da (Carr et al, (2002) Drug Discov Today. May 1; 7(9):522-7). These can then provide a starting point for medicinal chemistry to optimise the interactions using a structure-based approach. The fragments can be combined onto a template or used as the starting point for ‘growing out’ a ligand into other pockets of the protein (Blundell et al; Nat Rev Drug Discov. (2002) January; 1 (1):45-54). The fragments can be positioned in the binding pocket of the solAC and then ‘grown’ to fill the space available, exploring the electrostatic, van der Waals or hydrogen-bonding interactions that are involved in molecular recognition. The potency of the original weakly binding fragment thus can be rapidly improved using iterative structure-based chemical syntheses.

At one or more stages in the fragment growing approach, the ligand may be synthesized and tested in a biological system for its activity. This can be used to guide the further growing out of the fragment.

Where two fragment-binding regions are identified, a linked fragment approach may be based upon attempting to link the two fragments directly, or growing one or both fragments in the manner described above in order to obtain a larger, linked structure, which may have the desired properties.

The linked-fragment approach may be used for example to design ligands which occupy the ATP binding site and the bicarbonate binding site. Such a ligand which occupies at least part of both sites may have better selectivity for solAC than a ligand which only occupies the ATP biding site.

Where the binding site of two or more ligands are determined they may be connected to form a potential lead compound that can be further refined using e.g. the iterative technique of Greer et al. For a virtual linked-fragment approach see Verlinde et al. (Verlinde et al. (1992) J. of Computer-Aided Molecular Design, 6, 131-147), and for NMR and X-ray approaches see Shuker et al. (Shuker et al. (1996) Science, 274, 1531-1534) and Stout et al. (Stout et al. (1998) Structure, 6, 839-848). The use of these approaches to design solAC ligands is made possible by the determination of the solAC structure.

(vii) Compounds of the Invention.

Where a potential ligand for the solAC catalytic domain structure of the invention has been identified in accordance with the methods of the invention, the invention further includes the step of synthesizing the identified ligand and testing it in an in vivo or in vitro biological system in order to determine its activity and/or its effectiveness.

This aspect of the invention preferably further comprises the steps of:
obtaining or synthesizing the ligand; and
contacting the candidate ligand with solAC to determine the ability of the candidate ligand to interact with solAC.

For example, in the contacting step above the candidate ligand is contacted with solAC in the presence of a substrate, usually ATP, and typically a buffer, to determine the ability of said candidate ligand to bind to, e.g. inhibit, solAC. One such method is an HTRF based immunoassay, in which cAMP produced by solAC and XL-665-labelled cAMP compete for binding to an anti-cAMP antibody labelled with Eu-cryptate (http://www.htrf-assays.com; Gabriel D, Vernier M, Pfeifer M J, Dasen B, Tenaillon L, Bouhelal R. (2003) Assay & Drug Dev. Technol.; 2: 291-303). So, for example, an assay mixture for solAC may be produced which comprises the candidate ligand, substrate and buffer.

More preferably, in the latter step the candidate ligand is contacted with solAC under conditions to determine its function.

Alternatively, or in addition, the method may further comprise the steps of:
obtaining or synthesizing the ligand;
forming a complex of a solAC protein or catalytic domain thereof and said ligand; and
analysing said complex by X-ray crystallography to determine the ability of said ligand to interact with the solAC or catalytic domain thereof.

These steps may be used to identify ligands which can be fitted to the structure of the invention in an iterative process leading to the design of further ligands.

For example, in the contacting step above the candidate ligand is contacted with solAC in the presence of a substrate and typically a buffer, to determine the ability of said candidate ligand to bind to, e.g. inhibit, solAC. So, for example, an assay mixture for solAC may be produced which comprises the candidate ligand, substrate and buffer.

In another aspect, the invention includes a compound, which is a solAC ligand identified by the methods of the invention described herein.

Following identification of such a compound, it may be manufactured and/or used in the preparation, i.e. manufacture or formulation, of a composition such as a medicament, pharmaceutical composition or drug. These may be administered to individuals.

Thus, the present invention extends in various aspects not only to a compound as provided by the invention, but also a pharmaceutical composition, medicament, drug or other composition comprising such a compound. The compositions may be used for treatment (which may include preventative treatment) of disease such as cancer, inflammation, osteoporosis, diabetes, glaucoma or infertility. The compositions may also be used in contraceptive methods, e.g. to inhibit sperm motility or fertilization.

The term “treatment” as used herein in the context of treating a disease or condition, pertains generally to treatment and therapy, whether of a human or an animal (e.g. in veterinary applications), in which some desired therapeutic effect is achieved, for example, the inhibition of the progress of the condition, and includes a reduction in the rate of progress, a halt in the rate of progress, amelioration of the condition, and cure of the disease or condition. Treatment as a prophylactic measure (i.e. prophylaxis) is also included.

The term “therapeutically-effective amount” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

The term “treatment” includes combination treatments and therapies, in which two or more treatments or therapies are combined, for example, sequentially or simultaneously.

The compounds of, or obtained in accordance with, the invention include compounds which are inhibitors of solAC activity. As such, they are expected to be useful in providing a means of preventing and/or enabling sperm motility and hypermotility, capacitation and acrosome reaction. It is therefore anticipated that the compounds will prove useful in treating some forms of male infertility or sterility and/or preventing fertilisation of oocytes and consequently pregnancy.

Examples of cancers which may be treated include, but are not limited to, carcinomas, for example a carcinoma of the bladder, breast, colon (e.g. colorectal carcinomas such as colon adenocarcinoma and colon adenoma), kidney, epidermus, liver, lung, for example adenocarcinoma, small cell lung cancer and non-small cell lung carcinomas, oesophagus, gall bladder, ovary, pancreas e.g. exocrine pancreatic carcinoma, stomach, cervix, thyroid, prostate, or skin, for example squamous cell carcinoma; a hematopoietic tumour of lymphoid lineage, for example leukemia, acute lymphocytic leukemia, B-cell lymphoma, T-cell lymphoma, Hodgkin's lymphoma, non-Hodgkin's lymphoma, hairy cell lymphoma, or Burkett's lymphoma; a hematopoietic tumor of myeloid lineage, for example acute and chronic myelogenous leukemias, myelodysplastic syndrome, or promyelocytic leukemia; multiple myeloma; thyroid follicular cancer; a tumour of mesenchymal origin, for example fibrosarcoma or habdomyosarcoma; a tumor of the central or peripheral nervous system, for example astrocytoma, neuroblastoma, glioma or schwannoma; melanoma; seminoma; teratocarcinoma; osteosarcoma; xenoderoma pigmentoum; keratoctanthoma; or Kaposi's sarcoma.

Examples of inflammatory diseases or conditions ameliorated by the inhibition of solAC include, but are not limited to, rheumatoid arthritis, osteoarthritis, rheumatoid spondylitis, gouty arthritis, traumatic arthritis, rubella arthritis, psoriatic arthritis, and other arthritic conditions; Alzheimer's disease; toxic shock syndrome, the inflammatory reaction induced by endotoxin or inflammatory bowel disease; tuberculosis, atherosclerosis, muscle degeneration, Reiter's syndrome, gout, acute synovitis, sepsis, septic shock, endotoxic shock, gram negative sepsis, adult respiratory distress syndrome, cerebral malaria, chronic pulmonary inflammatory disease, silicosis, pulmonary sarcoisosis, bone resorption diseases, reperfusion injury, graft vs. host reaction, allograft rejections, fever and myalgias due to infection, such as influenza, cachexia, in particular cachexia secondary to infection or malignancy, cachexia secondary to acquired immune deficiency syndrome (AIDS), AIDS, ARC (AIDS related complex), keloid formation, scar tissue formation, Crohn's disease, ulcerative colitis, pyresis, chronic obstructive pulmonary disease (COPD), acute respiratory distress syndrome (ARDS), asthma, pulmonary fibrosis and bacterial pneumonia.

Of particular interest are compounds for use in the treatment or prophylaxis of inflammatory diseases and conditions, rheumatoid arthritis and osteoarthritis.

Thus the invention provides for the treatment of a condition mentioned above, wherein said treatment may comprise administration of a composition comprising a compound obtained according to the invention to a patient, e.g. for treatment of disease; the use of a compound, e.g. an inhibitor of solAC, in the manufacture of a composition for administration, e.g. for treatment of disease; and a method of making a pharmaceutical composition comprising admixing such a compound with a pharmaceutically acceptable excipient, vehicle or carrier, and optionally other ingredients.

Thus a further aspect of the present invention provides a method for preparing a medicament, pharmaceutical composition or drug, the method comprising: (a) identifying or modifying a compound by a method of any one of the other aspects of the invention disclosed herein; (b) optimising the structure of the compound; and (c) preparing a medicament, pharmaceutical composition or drug containing the optimised compound.

The above-described processes of the invention may be iterated in that the modified compound may itself be the basis for further compound design.

By “optimising the structure” we mean e.g. adding molecular scaffolding, adding or varying functional groups, or connecting the molecule with other molecules (e.g. using a fragment linking approach) such that the chemical structure of the ligand molecule is changed while its original modulating functionality is maintained or enhanced. Such optimisation is regularly undertaken during drug development programmes to e.g. enhance potency, promote pharmacological acceptability, increase chemical stability etc. of lead compounds.

Modification will be those conventional in the art known to the skilled medicinal chemist, and will include, for example, substitutions or removal of groups containing residues which interact with the amino acid side chain groups of a solAC structure of the invention. For example, the replacements may include the addition or removal of groups in order to decrease or increase the charge of a group in a test compound, the replacement of a charge group with a group of the opposite charge, or the replacement of a hydrophobic group with a hydrophilic group or vice versa. It will be understood that these are only examples of the type of substitutions considered by medicinal chemists in the development of new pharmaceutical compounds and other modifications may be made, depending upon the nature of the starting compound and its activity.

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington: The Science and Practice of Pharmacy”, 20th Edition, 2000, pub. Lippincott, Williams & Wilkins.

The invention is illustrated by the following examples:

### EXAMPLE

In order to obtain a polypeptide (or protein) that can be utilised for determination of the three dimensional (tertiary) structure of solAC, DNA encoding solAC may be obtained by total gene synthesis or by cloning. This DNA may then be expressed in a suitable expression system to obtain a polypeptide that can be subjected to techniques to determine its three dimensional structure.

Summary of Cloning, Expression and Purification of solAC.

In order to obtain expression and purification of solAC for structural studies, a number of different constructs were made, for expression in both E. coli and insect cells. In the detailed examples which follow, the expression of a preferred construct, 2056, is described. The conditions for the expression and recovery of this construct were arrived at after extensive investigation of numerous constructs and expression and purification conditions.

The constructs included all or part of the solAC protein between residues 1 and 469, as well as N-terminal or C-terminal tags. C-terminal tags were either Tobacco Etch Virus (TEV) Protease-cleavable-GST or TEV-cleavable-His6 tags. His6 tags were also used at the C-terminal in some constructs. The constructs analysed are set out in Table 14 below.

TABLE 14

| | Construct | Description of construct | |
|---|---|---|---|
| 1 | 2022 | TEV-Cleavable-GST--- | 1-469 |
| 2 | 2021 | TEV-Cleavable-GST--- | 1-469---His6 |
| 3 | 2023 | TEV-Cleavable-His6--- | 1-469 |
| 4 | 2056 | | 1-469 ---His6 |
| 5 | 2024 | TEV-Cleavable-His6--- | 13---469 |
| 6 | 2026 | TEV-Cleavable-His6--- | 36---469 |
| 7 | 2025 | TEV-Cleavable-His6--- | 27---469 |
| 8 | 2027 | | 1-469 |
| 9 | 2044 | TEV-Cleavable-His6--- | 1---245 |
| 10 | 2032 | | 255-469--- His6 |
| 11 | 2033 | TEV-Cleavable-His6 --- | 1-469 |
| 12 | | | 1-469 --- His6 |
| 13 | 2100 | TEV-Cleavable-His6--- | 13---469 |
| 14 | 2101 | TEV-Cleavable-His6--- | 36---469 |
| 15 | 2102 | TEV-Cleavable-His6--- | 27---469 |

Constructs 1-8 were for expression in a baculovirus expression system, and 9-15 for expression in E. coli.

E. coli Constructs

All of the constructs expressed as inclusion bodies when the temperature was maintained between 30 to 37° C. upon induction. Attempts to achieve soluble expression by lowering the temperature as far down as 15° C. upon induction resulted in some soluble expression, but severe proteolytic degradation of all constructs as observed by western blot analysis using anti SAC antibodies. Inclusion body preparations of construct 2033 were pursued using the T. brucei AC protocol of Bieger (Bieger, B. and Essen, L-O. (2000) Acta Cryst. D56, 359-362) as a starting point.

Briefly, cells (construct 2033 mainly) were suspended, lysed by sonication and clarified by centrifugation. The inclusion body pellets were re-suspended and extensively washed before re-solubilisation in either 6M GuHCl or 8M Urea. Buffers used were predominantly Tris HCl pH 8.0, Hepes, pH 8.0 or PBS, pH 7.6. The detergent used for some of the washes was Triton 100. Both Ni-NTA and the non-nickel, immobilized metal affinity chromatography (IMAC) resin TALON™ (Clontech, Mountain View, Calif.) columns were used under denaturing conditions to capture the protein of interest. Dialysis of the denatured solubilized proteins against a 10 mM NaH₂PO₄/Na₂HPO₄ pH 7.6, 0.3M NaCl, 0.4M Arginine, 10% glycerol for 2 hours, caused a large proportion of the contaminants to remain in solution, where as the protein of interest crashed out of solution. Re-solubilized solAC protein was used in a refolding screen of 8×96 conditions. A set of conditions were identified that resulted in refolded active protein, for example: 50 mM HEPES pH7.2, 0.5M Arginine, 0.3M NaCl, 5 mM BME and 10% Glycerol. This yielded folded active protein which could then be applied to a Ni-NTA column for further purification. Yields of protein were low and further study was focussed on insect cell expression.

### Baculovirus Constructs

Work focused predominantly around the constructs described in the literature as these had been shown to be active and competent in the assays. There were however significant concerns regarding the published low expression yields and lack of purification precedent. Expression of the solAC constructs was tested in Sf9 and High Five™ cells and shown to be better in the latter.

The N-terminally GST tagged solAC construct (2022) comprises an engineered TEV cleavage site in the linker between the GST and solAC domains. Cells were initially suspended in PBS, 10% glycerol, 2 mM BME lysed by sonication on ice, and clarified by centrifugation. Supernatant was re-clarified by centrifugation, as it turned translucent/opaque within minutes of pooling the tubes. SDS PAGE and western blot analysis of the pellet showed that intact and proteolytically cleaved sAC constituted a significant proportion of the precipitate. A strategy to stabilize the lysate was sought by examining a range of buffers at two temperatures 4° C. and room temperature (about 20° C.). These buffers are set out in Table 15 below.

TABLE 15

50 mM NaAcetate pH4.5
50 mM NaAcetate pH4.5; 0.1% Triton
50 mM NaAcetate pH4.5; 0.3M NaCl
50 mM NaAcetate pH4.5; 0.5M NaCl
50 mM Mes pH 6.5
50 mM Mes pH 6.5; 0.1% n-octyl gucopyranoside
50 mM HEPES pH 7.5
50 mM HEPES pH 7.5; 0.5M NaCl
50 mM HEPES pH 7.5; 1 mM lauryl maltoside
50 mM Tris pH 8.5
50 mM Tris pH 8.5; 0.5M NaCl
50 mM Tris pH 8.5; 0.1% n-octyl glucopyranoside

For all the buffers, ˜2g of cells were suspended in 5 ml of each of the buffers and put through a freeze thaw cycle to burst them open. The propensity of the suspensions to ‘cloud’ or precipitate was noted. Best results were obtained when pH was between 7.5 and 8.5, the temperature kept at 4° C. and in the presence of 0.3M or 0.5M NaCl. The detergents were seen to have a deleterious effect. Separate experiments with PBS suggested stability problems with this buffer.

Optimal conditions were found to be 50 mM Tris pH 7.5 (measured at 4° C.); 0.3M NaCl 10% glycerol; and 2-5 mM BME.

Construct 2056 has a C-terminal hexahistidine tag which allowed the use of Ni-NTA resin to capture the protein. Use of TALON™ columns did not improve capture. Though the choice of buffer helped significantly in stabilising the lysate, the need for a slow flow rate to allow enough contact time with the resin still caused difficulties which lead to the implementation of a pre-cleanup step by means of a DEAE column. For this, the cells were suspended in very low salt buffer, lysed, and clarified and applied to a DEAE column. The conductivity of the lysate had to be maintained very low, since the target protein bound weakly to the matrix. Failure to do this caused poor capture. Elution was done via a two step gradient, with both constructs 2022 and 2056 eluting in the first step. Most of the contaminants which readily precipitate failed to bind to this column. The fractions were pooled and NaCl concentration was adjusted to 300 mM before application on to either the Ni-NTA column or Glutathione sepharose 4b column. The addition of this step enabled slow flow rates in subsequent steps which led to higher recovery of enzyme. Later on we were also able to batch bind to Ni-NTA Fast flow resin directly after clarification, having lysed the cells in the high salt buffer.

Removal of the imidazole from the solAC-2056 containing fractions was a key component to the success of protein recovery. Once the reproducibility of the Ni-NTA elution was established, the protein pool was buffer exchanged into 50 mM Tris pH 7.5 (4° C.), 30 mM NaCl, 10% Glycerol and 1 mM BME in order to apply to a resource Q column. The residence time of the protein in low salt was kept to a minimum as the highest losses in protein yield were experienced at this step, with up to 50% of the protein being lost. It was observed that the resource Q step worked well though the pH and the conductivity of the buffers at this step were found to be of extreme importance. If either of these parameters was altered, the protein failed to bind or the purification profile on a gel showed no improvement. The final step of the purification served to place the protein in the most stable buffer we had discovered: 50 mM Tris pH 7.5 (4° C.), 330 mM NaCl, 10% Glycerol, 1 mM BME.

The protein could be concentrated without any evidence of aggregation to beyond 40 mg/ml, though crystallizations were set up at 10 mg/ml.

Surprisingly, despite the similarity of the 2022 and 2056 constructs (2022 post TEV cleavage only differed in a few amino acid residues at the N-terminus and the absence of the His6 tag at the C-terminus), 2022 oligomerised at concentrations of 5 mg/ml and higher, whereas 2056 remained monomeric up to 40 mg/ml.

Cloning, Expression and Purification of solAC-2056

An expression vector pcDNA3.1 encoding full length solAC (Nucleotide NM 018417 SwissProt) was used as a template for PCR amplification.

Cloning of solAC-2056

The construct M1-V469 was amplified using PCR.

The 5′ primer:

(SEQ ID NO: 1)

5′GGGGACAAGTTTGTACAAAAAAGCAGGCTCCATGAACACTCCAAAAGA
AGAATTCCAGGACTGG3′

carried an attB1 recognition sequence, and sequence corresponding to bases 1-11.

The 3′ primer:

(SEQ ID NO: 2)

5′GGGGACCACTTTGTACAAGAAAGCTGGGTTCAGTGGTGGTGGTGGTGG
TGGACTTTCTCAGTACGGCCC3′

introduces a sequence corresponding to bases 463-469, a 6 his c-terminal tag, a stop codon and an attB2 recognition site.

PCR Reaction reagents: 10 μl Thermopol buffer 10×, 2 μl dNTP mix, 1 μl DNA template, 1 μl 5′ primer, 1 μl 3′ primer, 1 μl Vent. Reaction made up to 100 μl with H2O.

PCR Cycling: 25 cycles of 94° C. for 30 sec, 55° C. 1 min, 72° C. for 3 mins, followed by an extension of 10 min at 72° C.

The 1407-bp fragment was recombined into the entry vector, pDONR using the BP reaction of the Gateway cloning technique.

An aliquot of the product was transformed into DH5α competent cells. Plasmid was extracted from the bacteria grown on the kanamycin selective plates. The sequence was confirmed by DNA sequencing before transferring the clone to the destination vector, pDEST8 via the LR reaction of the Gateway cloning technique. An aliquot of the product was transformed into DH5α competent cells. Plasmid was extracted from the bacteria grown on the carbenicillin selective plates. The sequence was confirmed by DNA sequencing before beginning expression of the protein.

The protein sequence of solAC-2056 is:

(SEQ ID NO: 3)

MNTPKEEFQDWPIVRIAAHLPDLIVYGHFSPERPFMDYFDGVLMFVDISG
FTAMTEKFSSAMYMDRGAEQLVEILNYHSAIVEKVLIFGGDILKFAGDAL
LALWRVERKQLKNIITVVIKCSLEIHGLFETQEWEEGLDIRVKIGLAAGH
ISMLVFGDETHSHFLVIGQAVDDVRLAQNMAQMNDVILSPNCWQLCDRSM
IEIESVPDQRAVKVNFLKPPPNFNFDEFFTKCTTFMHYYPSGEHKNLLRL
ACTLKPDPELEMSLQKYVMESILKQIDNKQLQGYLSELRPVTIVFVNLMF
EDQDKAEEIGPAIQDAYMHITSVLKIFQGQINKVFMFDKGCSFLCVFGFP
GEKVPDELTHALECAMDIFDFCSQVHKIQTVSIGVASGIVFCGIVGHTVR
HEYTVIGQKVNLAARMMMYYPGIVTCDSVTYNGSNLPAYFFKELPKKVMK
GVADSGPLYQYWGRTEKVHHHHHH

The 6 His tag is non cleavable in this construct.

Protein Production of solAC-2056

Production of recombinant virus of solAC-2056 was performed in the following manner. Briefly, the pDEST8 vector encoding the relevant gene was transformed into E. coli DH10BAC cells containing the baculovirus genome (Bacmid DNA). Via a transposition event in the cells, a region of the pDEST8 vector containing the gene and a gentamycin resistance gene including the baculovirus polyhedron promoter was transposed directly into the Bacmid DNA. By selection on gentamycin, kanamycin, tetracycline and Bluo-gal, resultant white colonies should contain recombinant bacmid DNA encoding the relevant gene. Bacmid DNA was extracted from a culture of white DH10BAC cells and transfected into Spodoptera frugiperda Sf9 cells grown in SF900 II serum free media following manufacturers instructions. Virus particles were harvested 72 hours post infection. A 1 ml aliquot of harvested virus particles was used to infect 100 mls of sf9 cells containing 1×10⁶ cells/ml. Cell culture medium was harvested 72 hours post infection.

Hi 5 insect cells were cultured in EX-Cell 405 (JRH) serum free media to a density of 1×10⁶ cells/ml. A 5 ml aliquot of viral stock was added to each litre of Hi 5 cells. The cultures were incubated at 27° C. for 48-72 hrs. Cells were harvested by centrifugation at 4000 rpm for 8 minutes. Pellets were frozen at −80° C.

Protein Purification of solAC-2056

All procedures were performed at 4° C. unless stated otherwise. Cell pellets were thawed on ice and re-suspended in lysis buffer (50 mM Tris pH 7.5, 300 mM NaCl, 10% Glycerol, 2 mM BME, protease inhibitor cocktail (Calbiochem). Cells were lysed by sonication and lysate was incubated with DNase 1 for 1 hour at 4° C. Lysate was clarified by centrifugation at 14,000 or 25,000 rpm for 1 hour. The clarified lysate was further clarified by centrifugation as in the previous step, then passed through a 0.45 μm filter before being applied to metal chelating matrix (GE Healthcare) pre-charged with Ni²⁺ in batch bind mode. The resin or matrix was poured into a column and the solAC protein was eluted by addition of lysis buffer containing 250 mM Imidazole. Fractions were analysed by SDS PAGE and those containing solAC protein were pooled. The pooled protein was buffer exchanged into low salt by application to a G25-desalting column equilibrated in 50 mM Tris pH 7.5, 30 mM NaCl, 10% Glycerol, 5 mM BME. The buffer exchanged solAC protein was then applied to a 6 ml Resource Q cation exchange (GE Healthcare) column and eluted using a gradient of 0-30% 1M NaCl over 20 column volumes. Fractions were analysed by SDS PAGE and those containing solAC protein were pooled and applied to a 26/60 superdex-75 gel filtration column pre-equilibrated in 50 mM Tris, pH7.5, 330 mM NaCl, 10% glycerol, 5 mM BME. Fractions were analysed by SDS PAGE. SolAC fractions were pooled and concentrated to a final concentration of ˜10 mg/ml using a vivaspin 2 centrifugal concentrator (HY).

Alternative Purification of solAC-2056

All procedures were performed at 4° C. unless stated otherwise. Cell pellets were thawed on ice and re-suspended in lysis buffer (50 mM Tris pH 7.5, 10% Glycerol, 2 mM BME, protease inhibitor cocktail (Calbiochem). Cells were lysed by sonication and lysate was incubated with DNase 1 for 1 hour at 4° C. Lysate was clarified by centrifugation at 14,000 or 25,000 rpm for 1 hour. The clarified lysate was further clarified by centrifugation as in the previous step, then passed through a 0.45 μm filter before being applied to a DEAE cation exchanger resin. The protein was eluted via a 15 and 30% step gradient of 1M NaCl. The 15% peak was pooled, the NaCl concentration of the pooled protein was raised to 300 mM and then applied to a metal chelating, usually a 5 ml Hi-trap, column (GE Healthcare) pre-charged with Ni²⁺ and equilibrated in 50 mM Tris pH 7.5, 300 mM NaCl, 10% Glycerol, 2 mM BME. The solAC protein was eluted by addition of equilibration buffer containing 250 mM Imidazole. Fractions were analysed by SDS PAGE and those containing solAC protein were pooled. The pooled protein was buffer exchanged into low salt by application to a G25-desalting column equilibrated in 50 mM Tris pH 7.5, 30 mM NaCl, 10% Glycerol, 5 mM BME. The buffer exchanged solAC protein was then applied to a 6 ml Resource Q cation exchange (GE Healthcare) column and eluted using a gradient of 0-30% 1M NaCl over 20 column volumes. Fractions were analysed by SDS PAGE and those containing solAC protein were pooled and applied to a 26/60 superdex-75 gel filtration column pre-equilibrated in 50 mM Tris, pH7.5, 330 mM NaCl, 10% glycerol, 5 mM BME. Fractions were analysed by SDS PAGE. SolAC fractions were pooled and concentrated to a final concentration of ˜10 mg/ml using a vivaspin 2 centrifugal concentrator (HY).

### Protein Crystallization of Soluble Adenylate Cyclase

A commercially available microbatch crystallization screen was used to grow crystals of human solAC using the sitting or hanging drop vapour diffusion method. Against a range of conditions, it was found that crystals grew from 200 mM citrate salt (eg sodium, potassium, ammonium) and 20% PEG3350. These crystals were chunky in shape but very small. Diffraction from these crystals was poor and reached a maximum resolution of 6.0 Angstroms. It was therefore necessary to find different conditions in which crystals of a size and acceptable resolution of diffraction could be made.

The crystals obtained in the initial screen grew from a solution which was not buffered. In further rounds of screening, using the hanging drop vapour diffusion method, it was found that using a buffer of pH 4.6-5.2 improved crystal growth.

The final pH of the solution against which the crystals were equilibrated varied from pH 6.0-6.4. In further round of screening, the citrate concentration was kept constant at 0.2M trisodium citrate, while the concentration and molecular weight of PEG was varied. It was found that using PEG 4000, at concentrations varied between 10-20%, improved crystallization.

The best crystals appeared at 16% PEG4K, however, their quality was not consistent, and their size varied from 0.05 mM to 0.2 mM in the longest dimension. The best of these crystals diffracted to a resolution of 2.3 Angstroms.

Thus further optimization was required. The protein was concentrated to ˜10 mg/ml in 50 mM Tris/HCl, pH7.5, 330 mM NaCl, 2 mM beta mercaptoethanol and 10% glycerol. The protein solution was mixed 1:1 by volume mixture with reservoir solution of 0.1M sodium acetate, pH 4.8, 0.2M trisodium citrate, 16-18% PEG4K and 10% glycerol. Crystals were grown at 4° C. by the method of hanging drop vapour diffusion.

Crystals appeared in the drops after 3-6 days and reached a maximum size of 0.5×0.1×0.1 mm after 14 days. Consistency of crystal size and quality was further improved using a microseeding method. The seed stock was prepared by crushing a solAC crystal in a solution of 0.1M sodium acetate, pH 4.8, 0.2M trisodium citrate, 14% PEG4K, 2 mM beta mercaptoethanol and 10% glycerol. The optimal dilution of the seed stock was reached by doing atrial crystallization run, in which a tray was set up using seed stocks of 1/100, 1/10000, 1/100,000 and 1/1000,000 dilutions.

The crystallization trays were set up by mixing equal volumes (1 μl+1 μl usually) of protein solution and seed stock on a cover slip. This was placed over a well containing 0.1M sodium acetate, pH 4.8, 0.2M trisodium citrate, 14% PEG4K and 10% glycerol The required dilution varied from batch to batch of protein.

The crystals grew in space group P6₃ with cell dimensions of a=b=99.15 Å, c=97.51 Å.

In order to preserve the crystals during data collection at low temperatures, crystals of solAC were briefly transferred to a cryobuffer solution containing 0.1M sodium acetate, pH 4.8, 0.2M trisodium citrate, 30% PEG4K and 10% glycerol. From this solution the crystals were plunged into liquid nitrogen and stored for subsequent data collection.

### Data Collection and Processing

Diffraction data used to solve the structure of solAC was collected in house using either a Jupiter CCD detector or an RAXIS HTC image plate detector. Both were mounted on Rigaku rotating anode generators. The high resolution data used to refine the solAC structure at 1.7 Å was collected on Beamline ID29-1 at the European Synchrotron Radiation facility, using an ADSC Quantum4 CCD detector, with a wavelength of 0.934 Å and processed using MOSFLM (Leslie, A. G. W. (1992). In Joint CCP4 and EESF-EACMB Newsletter on Protein Crystallography, vol. 26, Warrington, Daresbury Laboratory). The dataset was scaled using SCALA (CCP4—Collaborative Computational Project 4. (1994) The CCP4 Suite: Programs for Protein Crystallography. Acta Crystallographica D50, 760-763) and the intensities converted to structure factor amplitudes with TRUNCATE (Evans, P. R. (1997). Scaling of MAD data. In Recent Advances in Phasing (ed. K. S. Wilson, G. Davies, A. W. Ashton and S. Bailey), pp. 97-102. Council for the Central Laboratory of the Research Councils Daresbury Laboratory, Daresbury, UK), from the CCP4 suite of programs (CCP4—Collaborative Computational Project 4. (1994) The CCP4 Suite: Programs for Protein Crystallography. Acta Crystallographica D50, 760-763).

### Structure Determination and Refinement

The structure of soluble adenylate cyclase was solved using multiple isomorphous replacement. A wide variety of heavy metal solutions were prepared by dissolving compounds containing heavy metals in a solution consisting of 0.1M sodium acetate, pH 4.8, 0.2M trisodium citrate, 16% PEG4K and 10% glycerol. Crystals of solAC were then placed in the solution and equilibrated for varying lengths of time (2 minutes to 5 days). Many of these solutions caused damage to the crystals leading to effects from complete loss of diffraction to a lack of isomorphism of the soaked crystal with the original crystal.

Out of a large number of soaks (>100), 31 crystals gave useable datasets, and out of these 5 gave derivatives which could be analysed and solved for heavy atom position and occupancy. Difference Patterson methods were used to obtain initial positions for the heavy atoms, these were used for preliminary phasing with MLPHARE (CCP4 suite of programmes), difference Fourier maps were used to locate additional sites. This was repeated for the 5 useable derivatives, which were then cross checked using Difference Fouriers, to ensure that all solutions for heavy atoms were on the same origin.

Therefore 5 derivatives were used to obtain phases (shown in Table 16), which were then improved using solvent flattening ((Solomon) and cycles of ARP/WARP. The sequence of soluble adenylate cyclase was then built into the resulting electron density map. After multiple rounds of rebuilding and refinement (using QUANTA, REFMAC and CNX), initially at 2.3 Å using the in-house data and finally at 1.7 Å using the synchrotron data, the final soluble adenylate cyclase model consisted of residues 1-468, with 2 gaps (residues 135-140 and 350-356). The resulting structure is shown in Table 1.

TABLE 16
Heavy atom derivatives used to solve solAC structure

| | | Soak | | No of sites | Resolution/ | | Phasing |
|---|---|---|---|---|---|---|---|
| Compound | Concn/mM | time | | (Major/minor) | Angstrom | Riso | power |
| thimersal | 0.05 | 2.5 | hours | 4 | 2.5 | 0.18 | 0.98 |
| Potassium iodide | 150 | 2 | minutes | 2 | 2.5 | 0.24 | 0.62 |
| Dichloroethylene | Saturated | 5.5 | hours | 1 (3) | 2.2 | 0.12 | 0.57 |
| diamine platinum | | | | | | | |
| Ammonium | 1 | 3 | days | 4 | 2.8 | 0.24 | 0.66 |
| tertrabromoosmate | | | | | | | |
| Trimethyl lead | 10 | 3 | days | 3 | 3.0 | 0.22 | 0.89 |
| acetate | | | | | | | |

### Description of the Structure of Apo Soluble Adenylate Cyclase

The structure of the catalytic domain of soluble adenylate cyclase is described by the coordinates in Table 1. Residue Met1 is the first observable residue in the electron density and Lys468 is the last. An electron density peak close to the main chain nitrogen of Met1 may be due to acetylation of the N-terminus, and has not been modelled. Residues which have no interpretable main chain density are Trp135, Glu136, Glu137, Gly138, Leu139, Asp140, Phe350, Pro 351, Gly352, Glu353, Lys354, Val355 and Pro 356. Residue Val469 and the C-terminal His₆-tag are not visible in the electron density. The main chain close to the first break at 134 is poorly defined and residues 132-134 have patchy main chain electron density. Residues 304-306 and 451-455 are poorly defined by the electron density. In addition, several residues on the periphery of the model have had their side-chains placed arbitrarily as there is no interpretable density for them.

Active Site of solAC

Comparison of solAC with the enzyme from Spirulina platensis strongly suggests that solAC has only one active site, and this corresponds to the site formed by the A-chain residue 1140, the loop containing B-chain residue 1061 and the residues forming helix al from the B monomer in S. Platensis. Helix α1 does not exist for the second site which would correspond to the A monomer in S. platensis. Beta-strands corresponding to 2 and 3 in the A monomer are longer for solAC and partially obscure the adenosine binding site, which is a lot less open than the symmetry related corresponding site. Table 6 describes the residues in the active site region which are proposed to interact with the substrate.

### Preparation of Crystals of solAC Complexed with a Ligand

A solution of 200 mM α-β-methylene adenosine 5′-triphosphate, 40 mM CaCl₂ and 40 mM MnCl₂ was prepared in a soaking solution. The soaking solution consisted of 0.1M sodium acetate, pH 4.8, 0.2M trisodium citrate, 16% PEG4K, and 10% glycerol. A previously grown crystal of solAC catalytic domain was placed in 20 μL of the ligand soaking solution and allowed to equilibrate for 3 days. The crystal was then moved to a solution of cryoprotectant and frozen in liquid nitrogen in preparation for data collection.

### Data Collection, Structure Solution and Refinement for Ligand Complex

Diffraction data was collected for the solAC catalytic domain/ligand complex using an RAXIS HTC mounted on an in-house X-ray source. The previously solved structure of solAC catalytic domain was used as a starting model for refinement. Rigid body and restrained refinement was done and difference maps calculated. The ligand molecule, α-β-methylene adenosine 5′-triphosphate was built into the difference density and subsequent rounds of refinement done.

Description of the solAC Ligand Complex.

The structure of α,β methylene ATP complexed with solAC shows a mode of interaction which is very similar to that described for S. platensis. The majority of the interactions are between the phosphate groups, a metal ion and the protein. The adenine group forms a hydrogen bond between N6 amino group and the main chain carbonyl group of Val406, a water mediated interaction is formed between N7 and OD1 of Asn412. The ribose group makes no hydrogen bonds with the protein. The majority of the interactions are between the phosphate groups, a proposed metal binding site and the protein. Side chains of residues Asp99, Asp47, the carbonyl group of Ile48, oxygen atoms of the beta and gamma phosphates and a water molecule interact with the metal ion. Arg416 interacts with an oxygen from the alpha phosphate. Oxygen atoms from the gamma phosphate interact with OG of Thr52, and the main chain nitrogen atoms of Thr52 and Phe51.

On binding of the ligand, some rearrangement is seen in the region of residues 48-58 which corresponds to helix alpha 1 and loop joining beta 1 and alpha 1. The loop containing residues 96 to 101 undergoes a concerted motion so that the C-alpha of residue 99 moves 3.5 Å.

Further solAC-ligand Complexes
The HCO₃⁻ Binding Site of solAC

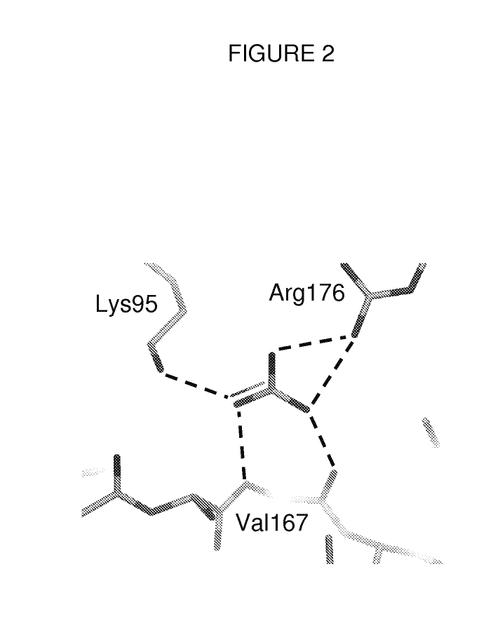

SolAC is the only protein whose activity is known to be directly regulated by HCO₃⁻. Soaking of solAC crystals with sodium bicarbonate has revealed the location of the HCO₃⁻ binding site. Binding of the HCO₃⁻ ion is mediated by a network of hydrogen bond and electrostatic interactions as shown in FIG. 2: 1) The NZ of Lys95 forms a salt bridge with the O1 of HCO₃⁻, 2) the backbone NH of Val167 forms a charged hydrogen bond with the O1 of HCO₃⁻, 3) the backbone carbonyl of Val167 forms a hydrogen bond with the OH of HCO₃⁻, 4) the sidechain NH2 of Arg176 forms a salt bridge with the O3 of HCO₃⁻ and 5) the sidechain NH2 of Arg176 forms a charged hydrogen bond with the OH of HCO₃⁻. The involvement of Lys95 in coordinating HCO₃⁻ is consistent with the conclusions drawn from a published study of bicarbonate-responsive adenylate cyclases (Cann, M. J. et al. (2003) Journal of Biological Chemistry 278, 35033-35038).

Binding of 5-Phenyl-2H[1,2,4]-triazole-3-thiol to the Bicarbonate Site of solAC

The structure of 5-Phenyl-2H-[1,2,4]-triazole-3-thiol (compound 1, purchased from Lancaster Synthesis, UK) bound to solAC reveals that the HCO₃⁻ binding site can be accessed by other small molecule ligands. The negatively charged sulphur atom of compound 1 sits in an almost identical position to the HCO₃⁻ ion, however the phenyl linked triazole group of compound 1 opens up the HCO₃⁻ binding site into a large pocket, contiguous with the ATP binding site. This expanded HCO₃⁻ binding site highlights a region of the solAC structure that can be exploited in the design of solAC inhibitors or activators.

The compound 1 mediated expansion of the HCO₃⁻ binding site is made possible by the movement of Arg176 out of the HCO₃⁻ pocket and the movement of a loop comprising Ala97, Gly98, Asp99 and Ala100. Compound 1 binding also induces a concerted movement in the sequence from Val335-Cys343. This region of solAC forms part of a beta strand and loop structure running along the bottom of the bicarbonate binding site. Phe336, Met337, and Phe338 in this sequence form part of the compound 1 binding site.

The expanded HCO₃⁻ binding site for compound 1 is lined by the following residues: Phe45, Leu94, Lys95, Ala97, Ala100, Leu102, Phe165, Leu166, Val 167, Ile168, Val172, Arg176, Val335, Phe336, Met337, and Phe338. The sulphur atom of compound 1 is located at the bottom of the pocket and forms a salt bridge with the NZ of Lys95 and charged hydrogen bonds with the backbone NH of Val167 and the backbone NH of Phe336. The N6 of the triazole also forms a hydrogen bond with the backbone carbonyl of Met337 at the bottom of the pocket. In addition, the N5 of the triazole forms a charged hydrogen bond with the NH2 of Arg176. The phenyl group of compound 1 extends into a predominantly lipophilic region of the compound 1 pocket formed by the sidechains of Phe45, Lys95, Ala97, Ala100, Leu102 and Phe336. Beyond the phenyl group of compound 1 the pocket narrows slightly before opening into the ATP binding site. The shape of the expanded HCO₃⁻ binding site and the nature of the interactions observed in the compound 1:solAC complex suggest that this site will be amenable to a variety of ligands.

Although there is a high level of sequence and structural similarity between mammalian transmembrane adenylate cyclase isoforms, solAC is the only member of this enzyme class that is regulated by HCO₃⁻. Thus, the structural information about the solAC HCO₃⁻ binding site described herein provides guidance for the design of solAC targeted drugs that are highly selective over tmAC's. Another critical feature of the compound 1 bound structure in regard to drug design is that the compound 1 exhibits clear growth vector opportunities out of the HCO₃⁻ site and into the ATP binding site of solAC. Compounds that target ATP binding sites of proteins are well precedented in drug discovery. The structures presented herein establish that compounds can be designed to occupy both the HCO₃⁻ and ATP binding of sites of solAC simultaneously.

Additional solAC Compound Binding Sites—N-(3-phenoxy-phenyl)-oxalamic acid

The crystal structure of N-(3-phenoxy-phenyl)-oxalamic acid (compound 2) bound to solAC reveals further regions of the solAC structure that might be exploited in drug design. The binding site for compound 2 overlaps with the expanded HCO₃⁻ pocket described for compound 1 such that the phenoxy group of compound 2 occupies a very similar position to the phenyl group of compound 1. However, compound 2 induces a large sidechain movement of Lys95, which lies at the bottom of the HCO₃⁻ binding site. This Lys95 movement opens up the HCO₃⁻ binding pocket to form a channel that merges with a large water filled cavity before opening onto the protein surface at a point opposite to the ATP binding site. The following residues line this newly exposed channel: His164, Phe165, Tyr268, Asn333, Lys334 and Val335. The oxalamic acid group of compound 2 protrudes furthest into this channel to form several interactions with the protein: 1) the compound 2 O3 forms a charged hydrogen bond with the ND of His164, 2) the compound 2 O1 forms a charged hydrogen bond with the backbone NH of Phe165, 3) the compound 2 O5 hydrogen bonds to the backbone NH of Val335 and 4) the amide N6 of compound 2 hydrogen bonds to the backbone carbonyl of Phe165. The sidechains of Lys95, Phe165, Leu166, Val167 and Phe336 form a lipophilic environment around the central anilino aromatic ring of compound 2. Although the terminal phenoxy group of compound 2 binds within the same lipophilic pocket described for compound 1, the environment of this pocket is slightly altered via a compound 2 induced movement of a loop comprising Met337, Phe338, Asp339, Lys340 and Gly341. This loop movement drags Phe338 away from the ATP site to within van der Waals distance of the terminal phenoxy group of compound 2. This movement of Phe338 is not observed in the structures of apo, AMP-PNP and compound 1 bound solAC. In fact, the structure of the AMP-PNP complex reveals that Phe338 forms one end of the ATP site sitting close to the hydroxyl groups of the AMP-PNP ribose. The repositioning of Phe338 creates a new sub-pocket adjacent to the ATP binding site. This new sub-pocket is lined by the following residues: Phe296, Met418, N298, Ser343, Phe336, Met337, Gly341, Asp339, Met419, Cys342, Ala415, Arg416, Met300, and Lys340.

Compounds that induce the formation of this ATP sub-pocket create additional drug design opportunities within the solAC active site. The fact that this ATP sub-pocket is induced by a compound binding within an expanded HCO₃⁻ site reinforces the view that the HCO₃⁻ site holds great potential for the development of drugs with a high level of selectivity over the non-bicarbonate responsive tmAC's.

Importantly, the four binding sites described herein are not mutually exclusive but can form a continuous solAC binding site that includes the expanded ATP and HCO₃⁻ pockets described. This entire binding site presents an attractive target for the development of small molecule solAC drugs.

### Preparation of N-(3-phenoxy-phenyl)-oxalamic acid

To a solution of 3-phenoxyaniline (276 mg, 1.4 mmoles) and triethylamine (0.2 ml, 1.4 mmoles) in tetrahydrofuran (3 ml) was added drop wise chloro-oxo-acetic acid ethyl ester (0.175 ml, 1.5 mmoles). The resultant solution was stirred at ambient temperature for 40 minutes. To the reaction mixture was added water (1.5 ml) and sodium hydroxide (72 mg, 1.8 mmoles). The reaction mixture was stirred at ambient temperature for a further 24 hours. The reaction mixture was partitioned between dichloromethane and hydrochloric acid (2N). The aqueous layer was washed with dichloromethane. The dichloromethane layers were combined and then washed with hydrochloric acid (2N) and water. The organics were dried (MgSO₄) filtered and the solvent removed by evaporation in vacuo to give N-(3-phenoxy-phenyl)-oxalamic acid as a white solid (286 mg, 74%).

### Preparation of solAC Crystals Complexed with HCO3−

A solution of 50 mM sodium bicarbonate was prepared in a soaking solution consisting of 0.1 M sodium acetate, pH 4.8, 0.2 M tri-sodium citrate, 16% PEG 4000, and 10% glycerol. A previously grown crystal of the solAC catalytic domain was placed in 20 μl of the bicarbonate soaking solution and allowed to equilibrate for 3 hours. The crystal was then moved to a solution of cryoprotectant and frozen in liquid nitrogen in preparation for data collection.

### Preparation of solAC Crystals Complexed with Compounds

A stock soaking solution was prepared at 90% of the final volume and comprised 200 mM NaCl, 200 mM tri-sodium citrate (pH 6.4), 15% w/v PEG 4000 and 15% v/v glycerol. The remaining 10% of the volume was topped up with either 1) water to give the harvesting solution or 2) a compound stock solution (in DMSO) to make the final soaking solution. The harvesting solution was used for temporary storage of solAC crystals after collecting them from hanging drops as well as a reservoir solution during soaking to prevent evaporation of the soaking solution. Compound 1 and compound 2 stock solutions were prepared at 0.25 M in DMSO. The pH of the stock 90% soaking solution was adjusted to pH 7.3 for compound 1 in order to facilitate binding of this compound.

The final soaking solutions for compounds 1 and 2 were prepared by mixing 36 μl of a “90% stock solution” with 4 μl of compound stock solution. This gave a final compound concentration of 25 mM.

### Soaking/Freezing Procedure for Compounds

Crystals were collected from hanging drops into 10 μl of fresh harvesting solution. The reservoir was also filled with 50-100 μl of harvesting solution. Soaking solutions (6 μl) were placed into wells and the corresponding reservoirs filled with 50 μl of harvesting solution. Crystals were distributed into the soaking solutions (1 crystal/compound). Wells were sealed and soaking was allowed to proceed at 20° C. for 6 min in the case of compound 1 and for 4.5 hr in the case of compound 2. Seals were cut and crystals were mounted on micromounts and frozen in liquid nitrogen.

### Compound Numbering

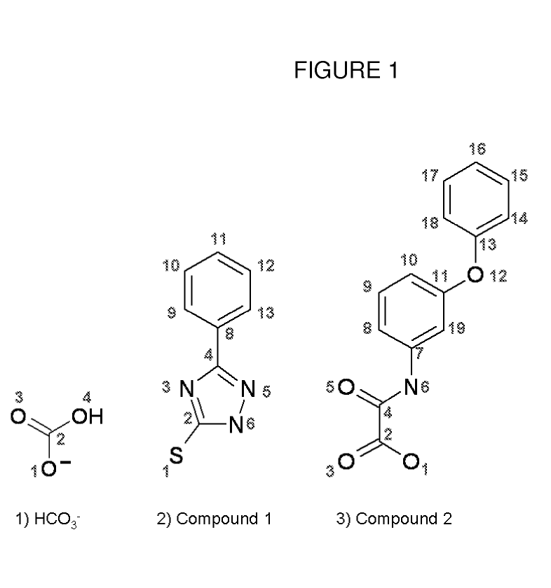

In the description of the solAC:HCO3−, solAC:compound 1 and solAC:compound 2 complexes the atom numbering scheme employed (as found in the associated pdb files) is shown in FIG. 1. Hydrogen atoms are not shown for compound 1 and compound 2. The hydrogen atom is shown for HCO₃₋ since this hydrogen is particularly important in defining the solAC:HCO3− interaction.

Lengthy table referenced here
US20090275047A1-20091105-T00001
Please refer to the end of the specification for access instructions.

Lengthy table referenced here
US20090275047A1-20091105-T00002
Please refer to the end of the specification for access instructions.

Lengthy table referenced here
US20090275047A1-20091105-T00003
Please refer to the end of the specification for access instructions.

Lengthy table referenced here
US20090275047A1-20091105-T00004
Please refer to the end of the specification for access instructions.

Lengthy table referenced here
US20090275047A1-20091105-T00005
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20090275047A1). An electronic copy of the table will also be available from the USPTO upon request and payment of the fee set forth in 37 CFR 1.19(b)(3).

We claim:

1. A computer-based method for the analysis of the interaction of a ligand with a solAC structure, which comprises:
providing a solAC structure which is of Table 1, Table 2, Table 3, Table 4 or Table 5, optionally varied by a root mean square deviation of not more than 1.5 Å, or selected coordinates thereof;
providing a ligand to be fitted to said solAC structure or selected coordinates thereof; and
fitting the ligand to said solAC structure.

2. The method of claim 1 wherein said selected coordinates include atoms from one or more of the groups of residues set out in any one of Tables 6, 7, 8, 9, 10 or 11.

3. The method of claim 1 wherein said selected coordinates include atoms from one or more of the amino acids from the groups Met1 to Tyr26 (SEQ ID NO: 7) or Lys219 to Gly284 (SEQ ID NO: 8).

4. The method of claim 2 wherein said ligand is fitted to at least one atom from at least 2, preferably at least 5, members of said group.

5. The method of claim 1 wherein said ligand is fitted to at least 10 atoms, preferably at least 100 atoms.

6. The method of claim 1 wherein the selected coordinates are of at least 500, preferably at least 1000 atoms.

7. The method of claim 1 wherein a plurality of molecular fragments are fitted and said fragments are assembled into a single molecule to form a ligand.

8. The method of claim 1 which further comprises the steps of:
obtaining or synthesising said ligand; and
contacting said ligand with a solAC protein to determine the ability of said ligand to interact with the solAC.

9. The method of claim 1 which further comprises the steps of:
obtaining or synthesising said ligand;
forming a complex of a solAC protein and said ligand; and
analysing said complex by X-ray crystallography to determine the ability of said ligand to interact with the solAC.

10. The method of claim 1 which further comprises the steps of:
obtaining or synthesising said ligand; and
determining or predicting how said ligand interacts with said solAC structure; and
modifying the ligand so as to alter the interaction between it and the solAC.

11. The method of claim 1 wherein the solAC structure is a model constructed from all or a portion of the coordinates of Table 1 Table 2, Table 3, Table 4 or Table 5, optionally varied by a root mean square deviation of not more than 0.5 Å, or selected coordinates thereof.

12. The method of claim 11 wherein the model is: (a) a wire-frame model; (b) a chicken-wire model; (c) a ball-and-stick model; (d) a space-filling model; (e) a stick-model; (f) a ribbon model; (g) a snake model; (h) an arrow and cylinder model; (i) an electron density map; j) a molecular surface model.

13. The method of claim 1 further comprising the step of:
(a) obtaining or synthesising the ligand; and
(b) contacting the ligand with solAC to determine the ability of the said ligand to interact with solAC.

14. A method of assessing the ability of a ligand to interact with solAC protein which comprises:
obtaining or synthesising said ligand;
forming a crystallized complex of a solAC protein and said ligand, said complex diffracting X-rays for the determination of atomic coordinates of said complex to a resolution of better than 3.5 Å; and
analysing said complex by X-ray crystallography by employing the data of Table 1, Table 2, Table 3, Table 4 or Table 5, optionally varied by a root mean square deviation of not more than 1.5 Å, or selected coordinates thereof, to determine the ability of said ligand to interact with the solAC protein.

15. A method according to claim 14 which comprises:
providing a crystal of the solAC protein;
soaking the crystal with the ligand to form a complex; and
determining the structure of the complex by employing the data of Table 1 Table 2, Table 3, Table 4 or Table 5, optionally varied by a root mean square deviation of not more than 1.5 Å, or selected coordinates thereof.

16. A method according to claim 14 which comprises:
mixing the solAC protein with the ligand;
crystallizing a solAC protein-ligand complex; and
determining the structure of the complex by employing the data of Table 1, Table 2, Table 3, Table 4 or Table 5, optionally varied by a root mean square deviation of not more than 1.5 Å, or selected coordinates thereof.

17. The method of claim 14 which further comprises determining the structure of said ligand.

18. The method of claim 14 which further comprises the steps of:
obtaining or synthesising the ligand; and
modifying the ligand so as to alter the interaction between it and the solAC.

19. A method for determining the structure of a protein, which method comprises;
providing the co-ordinates of Table 1, Table 2, Table 3, Table 4 or Table 5, optionally varied by a root mean square deviation of not more than 1.5 Å, or selected coordinates thereof, and
either (a) positioning said co-ordinates in the crystal unit cell of said protein so as to provide a structure for said protein, or (b) assigning NMR spectra peaks of said protein by manipulating said co-ordinates.

20. A method of predicting three dimensional structures of solAC protein homologues or analogues of unknown structure, the method comprises the steps of:
aligning a representation of an amino acid sequence of a target solAC protein of unknown three-dimensional structure with the amino acid sequence of the solAC of Table 1, Table 2, Table 3, Table 4 or Table 5, optionally varied by a root mean square deviation of not more than 1.5 Å, or selected coordinates thereof, to match homologous regions of the amino acid sequences;
modelling the structure of the matched homologous regions of said target solAC of unknown structure on the corresponding regions of the solAC structure as defined by Table 1, Table 2, Table 3, Table 4 or Table 5, optionally varied by a root mean square deviation of not more than 1.5 Å, or selected coordinates thereof; and
determining a conformation for said target solAC of unknown structure which substantially preserves the structure of said matched homologous regions.

21. A method of providing data for generating structures and/or performing optimisation of ligands which interact with solAC, solAC homologues or analogues, complexes of solAC with ligands, or complexes of solAC homologues or analogues with ligands, the method comprising:
(i) establishing communication with a remote device containing
(a) computer-readable data comprising atomic coordinate data of Table 1, Table 2, Table 3, Table 4 or Table 5, optionally varied by an rmsd of less than 1.5 Å, or selected coordinates thereof, said data defining the three-dimensional structure of solAC catalytic domain, or selected coordinates thereof;
(b) atomic coordinate data of a target adenylate cyclase homologue or analogue generated by homology modelling of the target based on the data (a);
(c) atomic coordinate data of a protein generated by interpreting X-ray crystallographic data or NMR data by reference to the data of Table 1, Table 2, Table 3, Table 4 or Table 5 and
(d) structure factor data derivable from the atomic coordinate data of (a) or (c); and
(ii) receiving said computer-readable data from said remote device.

22. The method of claim 21 wherein said computer-readable data is solAC atomic coordinate data of Table 1 Table 2, Table 3, Table 4 or Table 5, optionally varied by a root mean square deviation of not more than 1.5 Å, or selected coordinates thereof, and wherein the method further comprises:
providing a ligand to be fitted to the solAC atomic coordinate data of Table 1 Table 2, Table 3, Table 4 or Table 5, optionally varied by a root mean square deviation of not more than 1.5 Å, or selected coordinates thereof; and
fitting the ligand to the solAC structure.

23. The method of claim 1 wherein said selected coordinates include at least 5%, preferably at least 10%, Cα atoms.

24. The method of claim 23 wherein said rmsd is calculated by reference to said Cα atoms.

25. A computer system, intended to generate structures and/or perform optimisation of ligands which interact with solAC, solAC homologues or analogues, complexes of solAC with ligands, or complexes of solAC homologues or analogues with ligands, the system containing computer-readable data comprising one or more of:
(a) solAC co-ordinate data of Table 1, Table 2, Table 3, Table 4 or Table 5 said data defining the three-dimensional structure of solAC catalytic domain, or selected coordinates thereof;
(b) atomic coordinate data of a target adenylate cyclase protein generated by homology modelling of the target based on the coordinate data of Table 1, Table 2, Table 3, Table 4 or Table 5;
(c) atomic coordinate data of a target adenylate cyclase protein generated by interpreting X-ray crystallographic data or NMR data by reference to the co-ordinate data of Table 1, Table 2, Table 3, Table 4 or Table 5;
(d) structure factor data derivable from the atomic coordinate data of (b) or (c).
and
(e) atomic coordinate data of Table 1, Table 2, Table 3, Table 4 or Table 5, optionally varied by an rmsd of less than 1.5 Å, or selected coordinates thereof.

26. A computer system according to claim 25, wherein said selected coordinates include atoms from one or more of the groups of residues set out in any one of Tables 6, 7, 8, 9, 10 or 11.

27. The computer system of claim 26 wherein said selected coordinates are for at least one atom from at least 2, preferably at least 5, members of said group.

28. The computer system of claim 25 wherein said ligand is fitted to at least 10 atoms, preferably at least 100 atoms.

29. A computer system according to claim 25 comprising:
(i) a computer-readable data storage medium comprising data storage material encoded with said computer-readable data;
(ii) a working memory for storing instructions for processing said computer-readable data; and
(iii) a central-processing unit coupled to said working memory and to said computer-readable data storage medium for processing said computer-readable data and thereby generating structures and/or performing rational drug design.

30. A computer system according to claim 29 further comprising a display coupled to said central-processing unit for displaying said structures.

31. The use of a computer for producing a three-dimensional representation of a solAC structure or a solAC-ligand complex wherein the solAC structure is of Table 1, Table 2, Table 3, Table 4 or Table 5, optionally varied by a root mean square deviation of not more than 1.5 Å, or selected coordinates thereof, wherein said computer comprises:
(i) a machine-readable data storage medium comprising a data storage material encoded with machine-readable data, wherein said data comprise the structure of Table 1, Table 2, Table 3, Table 4 or Table 5, optionally varied by a root mean square deviation of not more than 1.5 Å, or selected coordinates thereof;
(ii) instructions for processing said machine-readable data into said three-dimensional representation.

32. The use of claim 31 wherein said selected coordinates are atoms of one of more the residues set out in any one of Tables 6, 7, 8, 9, 10 or 11.

33. A method of preparing a composition comprising identifying a ligand according to the method of claim 1 and admixing the molecule with a carrier.

34. A process for producing a medicament, pharmaceutical composition or drug, the process comprising: (a) identifying a ligand according to the method as defined in claim 1; and (b) preparing a medicament, pharmaceutical composition or drug containing the ligand.

35. A process for producing a medicament, pharmaceutical composition or drug which comprises (a) identifying a ligand according to the method as defined in claim 1; (b) optimising the structure of the ligand; and (c) preparing a medicament, pharmaceutical composition or drug containing the optimised ligand.

36. A crystal of solAC catalytic domain.

37. A co-crystal of solAC catalytic domain and a ligand.

38. A co-crystal of solAC catalytic domain and a ligand having a space group P6₃.

39. The co-crystal of claim 32 having unit cell dimensions a=b=99.5 Å, c=97.4 Å, and α=β=90.00., γ=120.00, with a unit cell variability of 5% in all dimensions.

40. A crystal of solAC protein having a resolution of 3.5 Å or better.

41. A crystal of solAc protein having the structure defined by the co-ordinates of Table 1, Table 2, Table 3, Table 4 or Table 5, optionally varied by an rmsd of less than 1.5 Å.
